(19) United States
(12) Patent Application Publication
(10) Pub. No.: US 2019/0183670 A1
(43) Pub. Date: Jun. 20, 2019

(54) MOUTHPIECE

(71) Applicant: Mitsui Chemicals, Inc., Minato-ku, Tokyo (JP)

(72) Inventors: Hideyuki NAGAI, Iwakuni-shi, Yamaguchi (JP); Takashi YUKITA, Chiba-shi, Chiba (JP); Maki YAMAMOTO, Sodegaura-shi, Chiba (JP); Yasufumi TSUCHIYA, Funabashi-shi, Chiba (JP); Walid RAAD, Brooklyn, NY (US)

(73) Assignee: Mitsui Chemicals, Inc., Minato-ku, Tokyo (JP)

(21) Appl. No.: 16/311,303

(22) PCT Filed: Jun. 21, 2017
(86) PCT No.: PCT/JP2017/022916

§ 371 Date: Dec. 19, 2018

(30) Foreign Application Priority Data
Jun. 28, 2016 (JP) 2016-127904
Jun. 28, 2016 (JP) 2016-127905
Nov. 17, 2016 (JP) 2016-224519

(51) Int. Cl. A61F 5/56

(52) U.S. Cl. CPC A61F 5/566; A61C 7/36

(57) ABSTRACT

A mouthpiece includes a maxillary piece that is mounted to maxillary dentition, a mandibular piece that is mounted to mandibular dentition, a coupling member that positions the mandibular piece with respect to the maxillary piece, and a spacer. The spacer includes an anchor portion that includes an indentation and that is formed at an inner wall face of the maxillary piece, a protrusion that is fit into the indentation of the anchor portion, and a clamped portion that is clamped between the maxillary piece and the mandibular piece.

### TECHNICAL FIELD

The present disclosure relates to a mouthpiece.

### BACKGROUND ART

Hitherto, medical mouthpieces have been employed in the treatment of sleep apnea syndrome, temporomandibular joint disorders, and the like. Normally a gap naturally arises between upper and lower teeth in a closed mouth state, and there are individual patient-related differences in the gap between the upper and lower teeth. There are accordingly known methods, for example, to adjust the gap between a maxillary piece and a mandibular piece of a mouthpiece by employing a vertical adjustment member that is bitten between anterior portions of the maxillary and mandibular pieces.

Moreover, a mouthpiece is disclosed in Non-Patent Document 1 that enables a gap between a maxillary piece and a mandibular piece of a mouthpiece to be adjusted by fixing a vertical adjustment member to an opposing face (namely, to an occlusal surface) of the maxillary piece opposing the mandibular piece by using screws.

Moreover, a dental brace is disclosed, for example as in Patent Document 1, that includes a maxillary brace that fits to maxillary dentition, a mandibular brace that fits to mandibular dentition, and a bite system that connects the maxillary brace and the mandibular brace together. Furthermore, a mouthpiece is disclosed in Non-Patent Document 2 that has a bite adjustment tool to adjust the vertical dimension of occlusion.

### LIST OF DOCUMENTS

Patent Document 1: Specification of US Patent Application Publication No. 2007/0224567.
Non-Patent Document 1: “B.O.S.S. Bite Opening Smart Shims” by Tompkins Dental Supply Inc., AADSM 25th Annual Meeting, Jun. 10, 2016.
Non-Patent Document 2: pages 28 to 34 of “Dental Sleep Practice, spring 2016”, Mar. 1, 2016.

### SUMMARY OF INVENTION

### Technical Problem

In cases in which a vertical adjustment member is bitten by anterior parts of a maxillary piece and a mandibular piece, measures need to be taken to prevent dropout of the vertical adjustment member, such as exposing an anchor portion for anchoring the vertical adjustment member at a labial side, making the exposed anchor portion larger than the bitten portion, and the like. There is accordingly a concern regarding a detrimental effect on an appearance, and also regarding a feeling of discomfort arising due to the anchor portion, to which the height adjustment member is anchored, abutting inside faces of the cheeks and lips.

Moreover, as disclosed in Non-Patent Document 1, in cases in which the vertical adjustment member is fixed to the opposing face of the maxillary piece, a thickness of the opposing face needs to be increased in order to form an anchor portion (namely female threaded hole) at the opposing face to fasten a screw.

Moreover, with a dental brace such as that disclosed in Patent Document 1, there is a concern that a gap between upper and lower dentition cannot be adjusted, and regarding a feeling of discomfort arising when wearing the dental brace due to the maxillary brace and the mandibular brace being held in a state abutting each other.

Furthermore, with the mouthpiece disclosed in Non-Patent Document 2, there is a concern regarding the bite adjustment tool dropping out from the mouthpiece due to the bite adjustment tool not being fixed to the mouthpiece. Moreover, the size of the bite adjustment tool is enlarged in order to prevent the bite adjustment tool from being swallowed by a patient if it were to drop out. This leads to a feeling of discomfort arising when the mouthpiece is being worn.

An object of a first aspect of the present disclosure is accordingly to provide a mouthpiece that enables an anchor portion of a vertical adjustment member to be suppressed from projecting out to an outer wall face side and an opposing face side of the mouthpiece.

Moreover, an object of a second aspect of the present disclosure is to provide a mouthpiece that enables a vertical adjustment member to be made more compact, while also suppressing dropout of the vertical adjustment member.

Moreover, an object of a third aspect of the present disclosure is to provide a mouthpiece that enables a relative position adjustment member including a vertical adjustment member to be made more compact, while also suppressing dropout of the relative position adjustment member.

### Solution to Problem

The following exemplary embodiments <1> to <9> are examples of the first aspect of the present disclosure.

The following exemplary embodiments <10> to <17> are examples of the second aspect of the present disclosure.

The following exemplary embodiments <18> to <28> are examples of the third aspect of the present disclosure.

<1> A mouthpiece including a maxillary piece that is mounted to maxillary dentition, a mandibular piece that is mounted to mandibular dentition, a positioning member that positions the mandibular piece with respect to the maxillary piece, an anchor base that includes an anchor portion and is formed at an inner wall face of at least one of the maxillary piece or the mandibular piece, and a vertical adjustment member that includes a mounting portion mounted to the anchor portion of the anchor base, and a clamped portion clamped between the maxillary piece and the mandibular piece.

According to the above configuration <1>, the mounting portion of the vertical adjustment member is mounted to the anchor portion of the anchor base, and the clamped portion of the vertical adjustment member is clamped between the maxillary piece and the mandibular piece. This enables a gap to be formed by the vertical adjustment member between the maxillary piece and the mandibular piece, while also suppressing dropout of the vertical adjustment member.

The anchor portion can be suppressed from projecting out to an outer wall face side and an opposing face side of the maxillary piece or the mandibular piece due to the anchor base being formed at the inner wall face of at least one of the maxillary piece or the mandibular piece.

<2> The mouthpiece described in <1>, wherein the anchor base is formed at a posterior side of a dentition of at least one of the maxillary piece or the mandibular piece.

Generally, a width of opposing faces is larger at a posterior side (for example, tooth positions 4 to 8) of the maxillary piece and the mandibular piece than an anterior side (for example, tooth positions 1 to 3) thereof, and bite closure force from the upper and lower teeth is accordingly more easily applied to the posterior side when the mouth is closed. Thus according to the above configuration in <2>, by forming the anchor base at the posterior side of the dentition of the maxillary piece or the mandibular piece, the vertical adjustment member is easily clamped between the maxillary piece and the mandibular piece, enabling dropout of the vertical adjustment member to be suppressed.

Furthermore, by mounting the vertical adjustment member at the posterior side of the dentition of the maxillary piece or the mandibular piece, obstruction of tongue movement by the vertical adjustment member when the mouth is closed can also be suppressed more than configurations in which the vertical adjustment member is mounted at an anterior side of the dentition.

<3> The mouthpiece described in <1 or <2>, wherein, in a mounted state of the mounting portion of the vertical adjustment member to the anchor portion of the anchor base, the vertical adjustment member has an asymmetrical shape with respect to a plane bisecting the vertical adjustment member between the inner wall face side and an outer wall face side of the maxillary piece or the mandibular piece.

According to the above configuration <3>, the vertical adjustment member is configured with an asymmetrical shape with respect to the plane bisecting the vertical adjustment member between the inner wall face side and the outer wall face side of the maxillary piece or the mandibular piece. This enables, for example, a higher degree of flexibility for the shape of the vertical adjustment member, such as by shaping the clamped portion of the vertical adjustment member to match jaw shape or the like.

Note that in the present specification “a plane bisecting the vertical adjustment member” indicates a plane that is orthogonal to the main plane of the clamped portion of the vertical adjustment member (namely, orthogonal to a plane opposing the opposing face of the maxillary piece or the mandibular piece) and that bisects a surface area of the main plane of the clamped portion.

<4> The mouthpiece described in <3>, wherein, in a mounted state of the mounting portion of the vertical adjustment member to the anchor portion of the anchor base, the mounting portion is provided at the vertical adjustment member at the inner wall face side of the maxillary piece or the mandibular piece.

According to the above configuration <4> due to the mounting portion being provided at the inner wall face side of the vertical adjustment member, the mounting portion can be better suppressed from projecting to the outer wall face side of the maxillary piece or the mandibular piece and interfering with a positioning member or the like than in configurations in which the mounting portion is provided at the center or outer wall face side of the vertical adjustment member.

<5> The mouthpiece described in any one of <1> to <4>, wherein the anchor portion of the anchor base is configured by an indentation, and the mounting portion of the vertical adjustment member is configured as a protrusion that fits into the indentation.

According to the above configuration <5>, the anchor portion is configured as the indentation and the mounting portion is a protrusion. This enables the vertical adjustment member to be easily anchored to the anchor base and dropout of the vertical adjustment member to be suppressed by fitting the protrusion into the indentation.

<6> The mouthpiece described in any one of <1> to <4>, wherein the mounting portion of the vertical adjustment member is configured as a through hole that a screw is inserted through, and the anchor portion of the anchor base is a female threaded hole that the screw, inserted through the through hole, is screwed into.

According to the above configuration <6>, the mounting portion is configured as the through hole, and the anchor portion is configured as the female threaded hole. This enables the vertical adjustment member to be easily anchored to the anchor base and dropout of the vertical adjustment member to be suppressed by screwing the screw, inserted through the through hole, into the female threaded hole.

<7> The mouthpiece described in <5>, wherein the indentation includes an opening portion at an opposing face of the anchor base, opposing at least one of the maxillary piece or the mandibular piece, and in plan view of the opening portion of the indentation, an imaginary line along a length direction of the opening portion intersects with a center line of a dentition of the maxillary piece or the mandibular piece.

When the mouthpiece is being worn, the air (namely, inhaled air and exhaled air) flows between the maxillary piece and the mandibular piece each time the patient breaths. According to the above configuration <7>, the imaginary line along the length direction of the opening portion of the indentation intersects with the center line of dentition, namely, intersects with a direction of airflow. This enables the protrusion of the vertical adjustment member, fitted into the indentation, to be better suppressed from dropping out from the indentation due to the airflow than configurations in which the imaginary line does not intersect with the center line.

<8> The mouthpiece described in <5>, wherein the indentation includes an opening portion at an inner wall face of the anchor base, and when the anchor base is viewed from an opposing face side of the maxillary piece or the mandibular piece where they oppose each other, an imaginary line along a length direction of the opening portion intersects with a center line of a dentition of the maxillary piece or the mandibular piece.

According to the above configuration <8> the imaginary line along the length direction of the opening portion of the indentation intersects with the center line of the dentition, namely with the direction of airflow. This enables the protrusion of the vertical adjustment member, fitted into the indentation, to be better suppressed from dropping out from the indentation due to the airflow than configurations in which the imaginary line does not intersect with the center line.

<9> The mouthpiece described in <5>, wherein the indentation includes an opening portion at an inner wall face of the anchor base, and an imaginary line along a length direction of the opening portion intersects with an imaginary plane parallel to an opposing face of the maxillary piece or the mandibular piece where they oppose each other.

According to the above configuration <9>, the imaginary line along the length direction of the opening portion of the indentation intersects with the imaginary plane parallel to the opposing face of the mouthpiece, namely, intersect with the airflow direction. This enables the protrusion of the vertical adjustment member, fitted into the indentation, to be better suppressed from dropping out from the indentation due to the airflow than configurations in which the imaginary line does not intersect with the imaginary plane.

<10> A mouthpiece including a maxillary piece that is mounted to maxillary dentition, a mandibular piece that is mounted to mandibular dentition, a positioning member that positions the mandibular piece with respect to the maxillary piece, an anchor portion that includes a mount and that is formed at at least one location or more at an outer periphery of at least one of the maxillary piece or the mandibular piece, or that is formed on an opposing face of the maxillary piece or the mandibular piece where they oppose each other, and a vertical adjustment member that is mounted to the mount of the anchor portion and that is clamped between the maxillary piece and the mandibular piece.

According to the above configuration <10>, the vertical adjustment member is mounted to the mount of the anchor portion. This enables the vertical adjustment member to be clamped between the maxillary piece and the mandibular piece, while also enabling dropout of the vertical adjustment member to be suppressed. Moreover, the gap between the maxillary piece and the mandibular piece can be adjusted by replacing the vertical adjustment member.

Furthermore, due to the mount of the anchor portion being formed at at least one location or more at the outer periphery of the maxillary piece or the mandibular piece, or the mount of the anchor portion being formed at the opposing face, there is accordingly a lower risk of being swallowed by the patient even if the vertical adjustment member were to drop out than configurations in which the mount is formed at an inner wall face of the maxillary piece or the mandibular piece. This enables the vertical adjustment member to be made more compact, as the vertical adjustment member does not needing to be enlarged.

Note that in the present specification “an outer periphery of the maxillary piece or the mandibular piece” indicates the outer wall face of the maxillary piece or the mandibular piece as well as a portion including a projection, stopper portion, or the like formed at the outer wall face.

<11> The mouthpiece described in <10>, wherein the mount is formed at at least one location or more at an outer side a line passing through width direction centers of the opposing face.

According to the above configuration <11>, the mount is disposed at the outer side of the line passing through the width direction centers of the opposing face of the maxillary piece or the mandibular piece, namely, at the outer wall face side of the maxillary piece or the mandibular piece. This enables a risk of being swallowed by the patient to be lowered even if the vertical adjustment member were to drop out than configurations in which the mount is disposed at an inner side of the line passing through the centers.

<12> The mouthpiece described in <10> or <11>, wherein the anchor base is formed at an outer wall face of the maxillary piece or the mandibular piece so as to project outer side than other portions of the outer wall face, and the anchor portion is formed at the anchor base.

According to the above configuration, due to the anchor portion being formed at the anchor base, that is formed projecting out from the outer wall face of the maxillary piece or the mandibular piece, the vertical adjustment member can be prevented from interfering with the dentition when the vertical adjustment member is mounted to the anchor portion.

<13> The mouthpiece described in any one of <10> to <12>, wherein the positioning member is attached to a posterior side of the dentition of the maxillary piece and the mandibular piece, and the anchor portion is formed at at least one location or more at an anterior side of the dentition of the maxillary piece or the mandibular piece from the positioning member.

According to the above configuration <13>, the positioning member is attached at the posterior side of the dentition of the maxillary piece and the mandibular piece, and the anchor portion is formed at the maxillary piece or the mandibular piece at the anterior side from the positioning member. Thus when the vertical adjustment member is mounted to the anchor portion, the vertical adjustment member can be prevented from interfering with the positioning member.

<14> The mouthpiece described in <10> or <11>, wherein the positioning member includes a wing portion that is formed at an outer wall face of one of the maxillary piece or the mandibular piece and extends in a direction toward another of the maxillary piece or the mandibular piece, and a stopper portion that is formed at an outer wall face of the other of the maxillary piece or the mandibular piece at a position at a posterior side from the wing portion and that restricts movement to the posterior side of the wing portion, and the mount of the anchor portion is formed at at least one location or more at a face of the stopper portion opposing the maxillary piece or the mandibular piece.

The above configuration <14> enables the mandibular piece to be positioned by the wing portion and the stopper portion so as not to move to the posterior side with respect to the maxillary piece. Moreover, due to the anchor portion being formed at the stopper portion, there is no need to form the anchor portion at the outer wall face or opposing face of the maxillary piece or the mandibular piece.

<15> The mouthpiece described in any one of <10> to <14>, wherein the anchor portion is configured as a through hole, and the vertical adjustment member includes a protrusion, a length of the protrusion in an extension direction being shorter than a depth of the through hole, and the protrusion being fit into the through hole.

According to the above configuration <15>, due to the anchor portion being configured as a through hole penetrating through the maxillary piece or the mandibular piece, cleaning is easier than configurations in which the anchor portion is bottomed. Moreover, due to the length in the extension direction of the protrusion being shorter than the depth of the through hole, the protrusion can be prevented from being exposed from the through hole and interfering with the dentition.

<16> The mouthpiece described in <15>, wherein the vertical adjustment member has left-right symmetry with respect to a plane bisecting the protrusion along the extension direction of the protrusion.

According to the above configuration <16>, due to the vertical adjustment member having left-right symmetry with respect to the plane bisecting the protrusion, for example, a common spacer can be employed on the left and right in cases in which vertical adjustment members are anchored to anchor portions formed at two locations at left and right of the dentition of the maxillary piece or the mandibular piece.

<17> The mouthpiece described in <15> or <16>, wherein a hooked portion employed for retention is formed at a leading end of the protrusion.

According to the above configuration <17>, due to the hooked portion, employed for retention, being formed at the leading end of the protrusion of the vertical adjustment member, the vertical adjustment member can be suppressed from dropping out from the through hole configuring the anchor portion.

<18> A mouthpiece including a maxillary piece that is mounted to maxillary dentition, a mandibular piece that is mounted to mandibular dentition, a wing portion that is provided at an outer wall face of one of the maxillary piece or the mandibular piece and that extends in a direction toward another of the maxillary piece or the mandibular piece, a stopper portion that is provided at an outer wall face of the other of the maxillary piece or the mandibular piece and that restricts movement to a posterior side of the dentition of the mandibular piece by abutting the wing portion, and a relative position adjustment member that is provided at the wing portion or the stopper portion to adjust a relative position of the maxillary piece and the mandibular piece.

According to the above configuration <18>, the mandibular piece can be positioned by the wing portion and the stopper portion so that movement to the posterior side of the dentition does not occur with respect to the maxillary piece. Moreover, the relative position of the maxillary piece and the mandibular piece can be adjusted by the relative position adjustment member provided at the wing portion or the stopper portion and clamped between the maxillary piece and the mandibular piece.

Moreover, the wing portion or the stopper portion that is provided with the relative position adjustment member is strongly fixed to the maxillary piece or the mandibular piece. This enables the size of the relative position adjustment member itself to be made smaller, as well as enabling dropout of the relative position adjustment member to be suppressed. Note that due to the wing portion and the stopper portion being provided at the outer wall faces of the maxillary piece and the mandibular piece, a risk of being swallowed by the patient can be lowered even if the relative position adjustment member were to drop out when the mouthpiece is being worn than in configurations in which the wing portion and stopper portion are provided on the inner wall face.

<19> The mouthpiece described in <18>, wherein the wing portion is provided at an outer wall face of the mandibular piece and extends toward the maxillary piece, and the stopper portion is provided at an outer wall face of the maxillary piece at a position at a posterior side from the wing portion.

According to the above configuration <19>, the mandibular piece can be positioned so that movement to a posterior side with respect to the maxillary piece does not occur due to the wing portion, provided at the mandibular piece, abutting the stopper portion located at the posterior side from the wing portion.

<20> The mouthpiece described in <18> or <19>, wherein at least a portion of the relative position adjustment member is clamped between the maxillary piece and the mandibular piece.

According to the above configuration <20>, the gap between the maxillary piece and the mandibular piece can be adjusted by the relative position adjustment member being clamped between the maxillary piece and the mandibular piece.

<21> The mouthpiece described in <20>, wherein the relative position adjustment member is formed integrally to the wing portion, and the wing portion is detachably attached to a wing attachment portion provided at the maxillary piece or the mandibular piece.

According to the above configuration <21>, the wing portion, formed integrally with the relative position adjustment member, is detachably attached to the wing attachment portion. This enables the gap between the maxillary piece and the mandibular piece to be adjusted by replacing the wing portion with another equipped with a relative position adjustment member of a different vertical dimension.

<22> The mouthpiece described in <21>, wherein the wing attachment portion is provided at an outer wall face of the maxillary piece or the mandibular piece.

According to the above configuration <22>, the wing attachment portion is provided at an outer wall face of the maxillary piece or the mandibular piece. This enables the vertical dimension of the wing portion itself to be adjusted by attaching wing portions of a different vertical dimension to the wing attachment portion.

<23> The mouthpiece described in <21> or <22>, wherein an attachment face for the wing portion at the wing attachment portion lies in substantially a same plane as an opposing face of the maxillary piece opposing the mandibular piece or an opposing face of the mandibular piece opposing the maxillary piece.

According to the above configuration <23>, the attachment face at the wing attachment portion is in substantially a same plane as the opposing face of the maxillary piece or the mandibular piece. This makes for easier cleaning of the attachment face at the wing attachment portion and the opposing face of the maxillary piece or the mandibular piece when maintenance is performed on the mouthpiece.

<24> The mouthpiece described in any one of <18> to <20>, wherein the relative position adjustment member is detachably attached to an anchor portion of the wing portion, and the wing portion is formed integrally to the maxillary piece or the mandibular piece.

According to the above configuration <24>, the relative position adjustment member is detachably attached to the anchor portion of the wing portion. This enables the relative position of the maxillary piece and the mandibular piece to be adjusted by replacing the relative position adjustment member.

<25> The mouthpiece described in <20>, wherein the relative position adjustment member is formed integrally to the stopper portion, and the stopper portion is detachably attached to the stopper attachment portion provided at the maxillary piece or the mandibular piece.

According to the above configuration <25>, the stopper portion formed integrally with the relative position adjustment member is detachably attached to the stopper attachment portion. This enables the gap between the maxillary piece and the mandibular piece to be adjusted by replacing the stopper portion with another equipped with a relative position adjustment member of a different vertical dimension.

<26> The mouthpiece described in <25>, wherein the stopper attachment portion includes a rail member provided at an outer wall face of the maxillary piece or the mandibular piece, and a rail groove that the rail member fits into is formed at the stopper portion.

According to the above configuration <26>, fitting the rail member of the stopper attachment portion into the rail groove of the stopper portion enables the stopper portion to be easily attached to or detached from the stopper attachment portion, and also enables the stopper portion to be suppressed from dropping out from the stopper attachment portion.

<27> The mouthpiece described in any one of <18> to <20>, wherein the relative position adjustment member is detachably attached to the anchor portion of the stopper portion, and the stopper portion is formed integrally to the maxillary piece or the mandibular piece.

According to the above configuration <27>, the relative position adjustment member is detachably attached to the stopper portion. This enables the relative position of the maxillary piece and the mandibular piece to be adjusted by replacing the relative position adjustment member.

<28> The mouthpiece described in <24> or <27>, wherein the anchor portion is provided at an abutting face of the wing portion abutting the stopper portion, or at an abutting face of the stopper portion abutting the wing portion, and at least a portion of the relative position adjustment member is clamped by the wing portion and the stopper portion.

According to the above configuration <28>, the relative position adjustment member being clamped by the wing portion and the stopper portion when the mouthpiece is being worn enables the relative position adjustment member to be suppressed from dropping out from the anchor portion. Moreover, the relative position adjustment member is easily attached to or detached from the anchor portion when the mouthpiece is not being worn.

### Advantageous Effects

The first aspect enables an anchor portion of a vertical adjustment member to be suppressed from projecting to an outer wall face side or an opposing face side of a mouthpiece.

The second aspect enables a mouthpiece to be provided in which dropout of a vertical adjustment member can be suppressed and the vertical adjustment member can be made more compact.

The third aspect enables a mouthpiece to be provided in which dropout of a relative position adjustment member including a vertical adjustment member can be suppressed and the relative position adjustment member can be made more compact.

### BRIEF DESCRIPTION OF DRAWINGS

FIG. 1 is a front view illustrating vertical adjustment members of a mouthpiece of a first exemplary embodiment of a first aspect in a pre-attachment state.

FIG. 2A is a front view illustrating the vertical adjustment members of the mouthpiece of the first exemplary embodiment of the first aspect in a post-attachment state.

FIG. 2B is bottom view of a maxillary piece of the mouthpiece of the first exemplary embodiment of the first aspect in a state viewed from an opposing face side.

FIG. 3A is a front view illustrating vertical adjustment members of a mouthpiece of a second exemplary embodiment of the first aspect in a pre-attachment state.

FIG. 3B is a front view illustrating the vertical adjustment members of the mouthpiece of the second exemplary embodiment of the first aspect in a post-attachment state.

FIG. 4A is a bottom view of a maxillary piece of the mouthpiece of the second exemplary embodiment of the first aspect in a state viewed from an opposing face side.

FIG. 4B is a cross-section taken along line A-A of FIG. 3B. FIG. 5A is a front view illustrating vertical adjustment members of a mouthpiece of a third exemplary embodiment of the first aspect in pre-attachment state.

FIG. 5B is a front view illustrating the vertical adjustment members of the mouthpiece of the third exemplary embodiment of the first aspect in a post-attachment state.

FIG. 6A is a front view illustrating vertical adjustment members of a mouthpiece of a first exemplary embodiment of a second aspect in a pre-attachment state.

FIG. 6B is a front view illustrating the vertical adjustment members of the mouthpiece of the first exemplary embodiment of the second aspect in a post-attachment state.

FIG. 7A is a front view illustrating a vertical adjustment member of a mouthpiece of a second exemplary embodiment of the second aspect in a pre-attachment state.

FIG. 7B is a front view illustrating the vertical adjustment member of the mouthpiece according to the second exemplary embodiment of the second aspect in a post-attachment state.

FIG. 8A is a front view illustrating vertical adjustment members of a mouthpiece according to a third exemplary embodiment of the second aspect in a pre-attachment state.

FIG. 8B is a side view illustrating the vertical adjustment member of the mouthpiece according to the third exemplary embodiment of the second aspect in a post-attachment state.

FIG. 9A is a perspective view illustrating a portion of a mouthpiece according to a modified example of the second aspect.

FIG. 9B is a perspective view illustrating a portion of a mouthpiece according to a modified example of the second aspect.

FIG. 9C is a perspective view illustrating a portion of a mouthpiece according to a modified example of the second aspect.

FIG. 10A is a front view illustrating relative position adjustment members of a mouthpiece according to a first exemplary embodiment of a third aspect in a pre-attachment state.

FIG. 10B is a side view illustrating a relative position adjustment member of the mouthpiece according to the first exemplary embodiment of the third aspect in a post-attachment state.

FIG. 11A is a side view illustrating a relative position adjustment member of a mouthpiece according to a second exemplary embodiment of the third aspect in a pre-attachment state.

FIG. 11B is a side view illustrating the relative position adjustment member of the mouthpiece according to the second exemplary embodiment of the third aspect in a post-attachment state.

FIG. 12A is a side view illustrating a relative position adjustment member of a mouthpiece according to a third exemplary embodiment of the third aspect in a pre-attachment state.

FIG. 12B is a side view illustrating the relative position adjustment member of the mouthpiece according to the third exemplary embodiment of the third aspect in a post-attachment state.

FIG. 13A is a side view illustrating a relative position adjustment member of a mouthpiece according to a fourth exemplary embodiment of the third aspect in a pre-attachment state.

FIG. 13B is a side view illustrating the relative position adjustment member of the mouthpiece according to the fourth exemplary embodiment of the third aspect in a post-attachment state.

FIG. 14A is a side view illustrating a relative position adjustment member of a modified example of a mouthpiece according to the second exemplary embodiment of the third aspect in a pre-attachment state.

FIG. 14B is a side view illustrating the relative position adjustment member of the modified example of a mouthpiece according to the second exemplary embodiment of the third aspect in a post-attachment state.

FIG. 15A is a side view illustrating a relative position adjustment member of a modified example of a mouthpiece according to the fourth exemplary embodiment of the third aspect in a pre-attachment state.

FIG. 15B is a side view illustrating the relative position adjustment member of the modified example of the mouthpiece according to the fourth exemplary embodiment of the third aspect in a post-attachment state.

### DESCRIPTION OF EMBODIMENTS

In the present specification, “an outer wall face side” indicates a buccal side (namely, an outer side) when a mouthpiece is being worn, and “an inner wall face side” indicates a lingual side (namely, an inner side) when the mouthpiece is being worn. Moreover, “an anterior side of dentition” indicates a front teeth (e.g. tooth positions 1 to 3) side, namely a labial side when the mouthpiece is being worn, and “a posterior side of dentition” indicates a back teeth (e.g. tooth positions 4 to 8) side, namely a palatal side when the mouthpiece is being worn.

Explanation follows regarding examples of a first aspect to a third aspect of the present disclosure, with reference to the drawings. The present disclosure is not limited to embodiments illustrated in the drawings. Note that sizes of members in each of the drawings are only schematic, and relative size relationships between members are not limited thereto.

First Aspect

A mouthpiece according to a first aspect of the present disclosure includes a maxillary piece that is mounted to maxillary dentition, a mandibular piece that is mounted to mandibular dentition, a positioning member that positions the mandibular piece with respect to the maxillary piece, an anchor base that includes an anchor portion, and that is formed at an inner wall face of at least one of the maxillary piece or the mandibular piece, and a vertical adjustment member that includes a mounting portion mounted to the anchor portion of the anchor base, and a clamped portion clamped between the maxillary piece and the mandibular piece.

In the first aspect, the anchor base is formed at the inner wall face of at least one of the maxillary piece or the mandibular piece. This enables the anchor portion to be suppressed from projecting out to an outer wall face side and an opposing face side from the maxillary piece or the mandibular piece.

Note that in the following first aspect, configuration referred to as “an anchor portion” in the disclosure of Japanese Patent Application No. 2016-224519 is referred to as “the anchor base” herein, and configuration referred to as “a mount” therein is referred to as “the anchor portion” herein.

### First Exemplary Embodiment

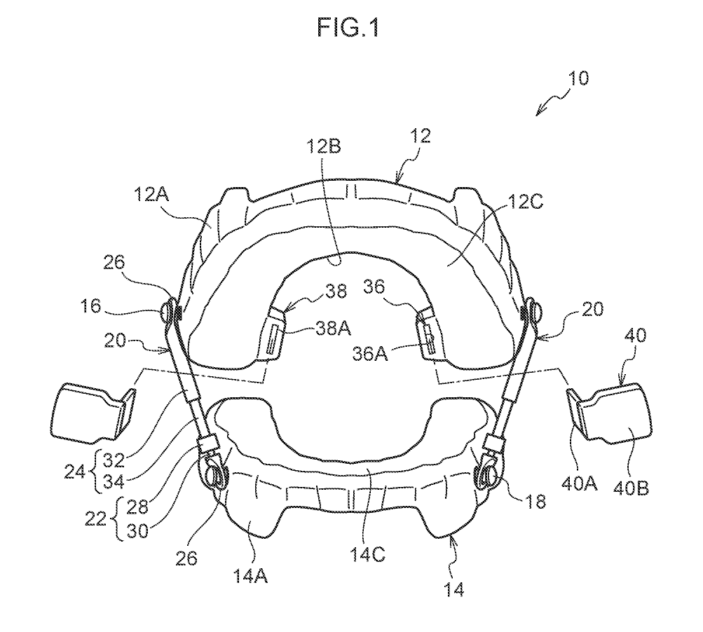

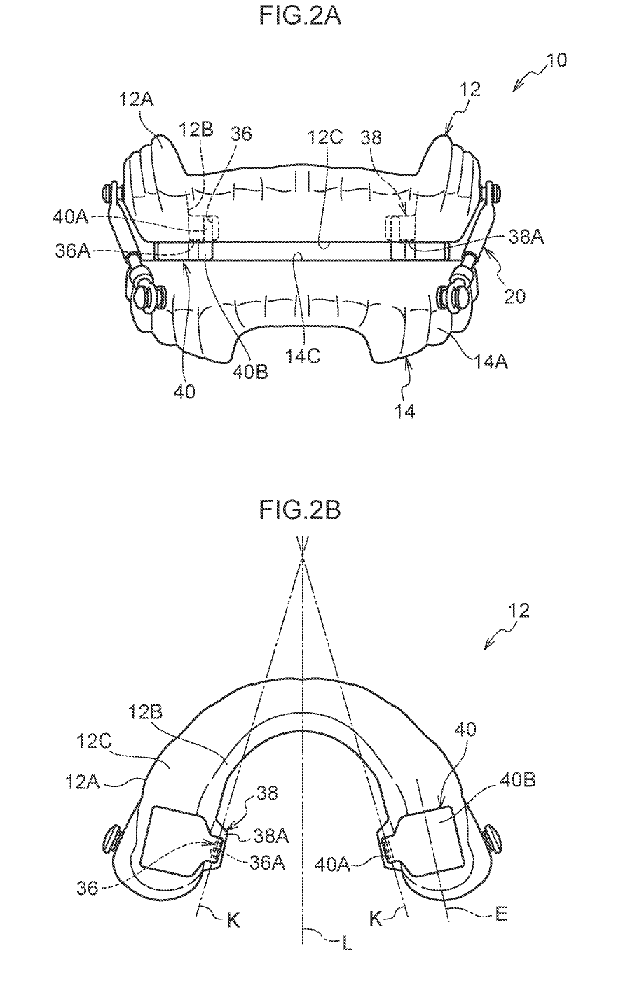

Explanation follows regarding a mouthpiece according to a first exemplary embodiment of the first aspect, with reference to FIG. 1 to FIG. 2B.

A mouthpiece 10 of the present exemplary embodiment is, for example, a mouthpiece employed for sleep apnea syndrome so as to reduce or prevent snoring and bruxism, and breathing interruptions or the like while sleeping. As illustrated in FIG. 1, the mouthpiece 10 includes a maxillary piece 12 worn at maxillary dentition, and a mandibular piece 14 worn at mandibular dentition.

The maxillary piece 12 and the mandibular piece 14 may, for example, be configured from an acrylic-based resin having a modulus of elasticity of approximately 2150 MPa in bending tests according to JIS T6501. Examples of resin materials that may be employed for the maxillary piece 12 and the mandibular piece 14 also include polyester-based resins, polycarbonate-based resins, urethane-based resins, vinyl acetate-based resins, polyamide-based resins, and the like.

Metal upper attachment portions 16 are provided at an outer wall face 12A of the maxillary piece 12, so as to project out from the outer wall face 12A at left and right posterior sides (tooth positions 6 to 7 in the present exemplary embodiment) as viewed from a center of a dentition. Metal lower attachment portions 18 are provided at an outer wall face 14A of the mandibular piece 14, so as to project out from the outer wall face 14A at left and right posterior sides (at tooth positions 3 to 4 in the present exemplary embodiment) as viewed from a center of a dentition.

The right upper attachment portion 16 and the right lower attachment portion 18, and the left upper attachment portion 16 and the left lower attachment portion 18, are respectively coupled together by metal coupling members 20 capable of pivoting. The coupling members 20 are positioning members to position the mandibular piece 14 so that movement to a posterior side does not occur with respect to the maxillary piece 12. The maxillary piece 12 and the mandibular piece 14 are coupled together by the pair of coupling members 20 so as to be capable of opening and closing.

In the mouthpiece 10 of the present exemplary embodiment, the upper attachment portion 16 is at the posterior side from (namely, at the posterior dentition side of) the lower attachment portion 18 as viewed from the center of dentition. Namely, the mouthpiece 10 is configured as a push-type mouthpiece that when worn pushes the mandibular piece 14 (and hence the mandible) forward using the coupling members 20.

The coupling members 20 each includes a length adjustment mechanism 22, a sliding mechanism 24, and eyelets 26 attached to the upper attachment portion 16 and the lower attachment portion 18. The length adjustment mechanisms 22 each includes a nut 28 and a male screw 30 screwed into the nut 28. A screw amount of the male screw 30 is adjusted by rotating the nut 28 to adjust a length of each coupling member 20 in a continuous manner.

The sliding mechanisms 24 each includes a sleeve 32 and a rod 34. The coupling members 20 are able to follow the maxillary piece 12 and the mandibular piece 14 by the rods 34 sliding inside the sleeves 32 to match movement of the maxillary piece 12 and the mandibular piece 14. A distance between the upper attachment portion 16 and the lower attachment portion 18 (a distance between respective centers) is adjusted by the coupling members 20 so as to be between about 18 mm and about 50 mm.

Anchor bases 38 including anchor portions 36 are respectively formed so as to project out from an inner wall face 12B at the left and right sides (at tooth positions 6 to 7 in the present exemplary embodiment) of the maxillary piece 12 as viewed from a center of dentition. The anchor portions 36 each is formed as a substantially rectangular shaped recess, and includes an elongated opening portion 36A at a lower face of the anchor base 38, namely, at an opposing face 38A that opposes the mandibular piece 14.

As illustrated in FIG. 2B, an imaginary line K, running along a length direction of each of the opening portions 36A when viewing the opening portions 36A of the anchor portion 36 in plan view, extends in a direction approaching a dentition center line L of the maxillary piece 12 on progression from the posterior side toward the anterior side of the dentition. The imaginary lines K each intersects with the center line L. Namely, a length direction (imaginary line K) of each of the opening portions 36A of the anchor portions 36 is not parallel to the center line L.

Note that angles of the imaginary lines K illustrated in FIG. 2B are an example thereof. The imaginary lines K may each be at any angle not parallel to the center line L. However, an angle formed between each of the imaginary lines K and the center line L is preferably from 5 degrees to 45 degrees, is more preferably from 5 degrees to 30 degrees, and is still more preferably from 5 degrees to 20 degrees.

In the present exemplary embodiment, as illustrated in FIG. 2A, the anchor portions 36 are configured by through holes that are also open at upper faces of the anchor bases 38. Moreover, the opposing face 38A of each of the anchor bases 38 lies in substantially the same plane as an opposing face 12C (namely, occlusal surface) of the maxillary piece 12 opposing the mandibular piece 14.

As illustrated in FIG. 1, a vertical adjustment member 40 configured, for example, from a resin material is mounted to each of the anchor portions 36. The vertical adjustment members 40 each includes a protrusion 40A serving as a mounting portion, and a clamped portion 40B that extends from one end of the protrusion 40A in a direction orthogonal to the extension direction of the protrusion 40A. The other end side of each of the protrusions 40A fits into the opening portion 36A of the anchor portion 36. Note that a retention means such as a hooked portion may be provided at the other end side of the protrusion 40A to maintain a state fitted into the anchor portion 36.

As illustrated in FIG. 2B, in a state in which the vertical adjustment member 40 is mounted to the anchor portion 36, the protrusion 40A of the vertical adjustment member 40 is provided further to an inner wall face side than a plane E bisecting the vertical adjustment member 40 between the inner wall face side and the outer wall face side. Namely, the vertical adjustment member 40 has an asymmetrical shape with respect to the plane E. The vertical adjustment members 40 each preferably has left-right symmetry with respect to a plane bisecting the protrusion 40A and the clamped portion 40B along an extension direction of the protrusion 40A.

The clamped portion 40B of each of the vertical adjustment members 40 has a substantially rectangular profile and a thickness normally from about 1 mm to about 10 mm. An extension length (namely a width) of the clamped portion 40B is either substantially the same as a width of the opposing face 12C of the maxillary piece 12 (namely, a length from the outer wall face 12A to the inner wall face 12B), or smaller than the width of the opposing face 12C.

When the protrusion 40A of each of the vertical adjustment members 40 has been fitted into the opening portion 36A of the anchor portion 36, the clamped portion 40B of the vertical adjustment member 40 is mounted to the opposing face 12C of the maxillary piece 12. Thus in a closed mouth state, as illustrated in FIG. 2A, the clamped portion 40B of each of the vertical adjustment members 40 is clamped between the opposing face 12C of the maxillary piece 12 and an opposing face 14C of the mandibular piece 14 that opposes the maxillary piece 12. A gap therefore arises between the maxillary piece 12 and the mandibular piece 14.

According to the present exemplary embodiment, by fitting the protrusion 40A of the vertical adjustment member 40 into the anchor portion 36 of the anchor base 38 formed at the maxillary piece 12, the vertical adjustment member 40 can easily be anchored to the maxillary piece 12, while also suppressing the vertical adjustment member 40 from dropping out. Moreover, due to the vertical adjustment member 40 being detachably attached to the anchor portion 36, the gap between the maxillary piece 12 and the mandibular piece 14 can be appropriately adjusted by replacing the vertical adjustment members 40 with others equipped with clamped portions 40B of a different thickness.

Specifically, for example, by preparing plural vertical adjustment members 40 with different thicknesses of clamped portions 40B by 1 mm, the gap between the maxillary piece 12 and the mandibular piece 14 can be adjusted by 1 mm by selecting the vertical adjustment member 40 that matches a gap between the upper and lower teeth of a patient. Note that a configuration may be adopted in which a gap is not formed (namely, the thickness is 0 mm) by anchoring vertical adjustment members 40 that only include the protrusions 40A into the anchor portions 36.

Moreover, the present exemplary embodiment enables the anchor bases 38 to be suppressed from projecting out to the outer wall face 12A side or the opposing face 12C side of the maxillary piece 12 due to the anchor bases 38 being formed at the inner wall face 12B of the maxillary piece 12.

Moreover, in the present exemplary embodiment the anchor bases 38 are formed at the posterior side of the dentition of the maxillary piece 12. Generally, the width (surface area) of opposing faces 12C, 12D is larger at the posterior side of the maxillary piece 12 and the mandibular piece 14 than at the anterior side thereof. Bite closure force from the upper and lower teeth is accordingly more easily applied at the posterior side of the dentition of the maxillary piece 12 and the mandibular piece 14 when the mouth is closed.

Thus by forming the anchor bases 38 at the posterior side of the dentition of the maxillary piece 12, the vertical adjustment members 40 are easily clamped between the maxillary piece 12 and the mandibular piece 14, enabling dropout of the vertical adjustment members 40 to be suppressed. Moreover, as well as being able to suppress the clamped portion 40B of each of the vertical adjustment members 40 from projecting to the outer wall face side of the maxillary piece 12 (namely, an outer side of the outer wall face 12A), obstruction of tongue movement by the vertical adjustment members 40 when the mouth is closed can also be suppressed.

In order to provide a given gap between the maxillary piece 12 and the mandibular piece 14 using the vertical adjustment members 40, the thickness of the clamped portions 40B, when the clamped portions 40B are clamped at the posterior side of the dentition, is thinner than the thickness of the clamped portions 40B, when the clamped portions 40B are clamped at the anterior side of the dentition.

Specifically, for example, a gap of from 4 mm to 5 mm can be disposed at the anterior side of the dentition by clamping clamped portions 40B having a thickness of 2 mm at the posterior side of the dentition. This enables a reduction in types of vertical adjustment member 40 required (namely, vertical adjustment members 40 having clamped portions 40B of a different thickness), enabling a reduction in cost.

Moreover, in the present exemplary embodiment the vertical adjustment members 40 have asymmetrical shapes with respect to the plane E, and the protrusions 40A are provided at the inner wall face side of the plane E. The protrusions 40A can thereby be suppressed from projecting out to the outer wall face side of the maxillary piece 12 (an outer side of the outer wall face 12A) and from interfering with the coupling members 20 better than configurations in which the protrusions 40A are provided at the center of the vertical adjustment members 40 or at the outer wall face side thereof.

Moreover, the width and shape of the clamped portions 40B of the vertical adjustment members 40 can be more freely set with respect to a position of the protrusions 40A than in configurations in which the vertical adjustment members 40 are symmetrically shaped with respect to the plane E. This enables, for example, the clamped portions 40B to be shaped to match the width and shape of the opposing face 12C of the maxillary piece 12, jaw shape, or the like.

Moreover, when the mouthpiece 10 is being worn, air (namely, inhaled air and exhaled air) flows between the maxillary piece 12 and the mandibular piece 14 along the center line L each time the patient breaths. Thus in the present exemplary embodiment, the length directions of the opening portions 36A of the anchor portions 36 formed at the opposing faces 38A of the anchor bases 38 are not parallel to the center line L, namely, not parallel to the airflow direction.

This enables the protrusions 40A of the vertical adjustment members 40 fitted into the anchor portions 36 to be suppressed from dropping out from the anchor portions 36 due to the airflow better than in configurations in which the length directions of the opening portions 36A of the anchor portions 36 are parallel to the center line L.

Moreover, in the present exemplary embodiment, the pair of anchor bases 38 are provided at the inner wall face 12B of the maxillary piece 12, and the gap between the maxillary piece 12 and the mandibular piece 14 is adjusted by the pair of vertical adjustment members 40. This enables play between the maxillary piece 12 and the mandibular piece 14 to be suppressed better than configurations in which the gap is adjusted using a single vertical adjustment member 40.

Moreover, in the present exemplary embodiment, due to the anchor portions 36 of the anchor bases 38 being configured by through holes that are open at the opposing face 38A (namely, the lower face) and at the upper face of the anchor bases 38, cleaning the inside of the anchor portions 36 is easier than configurations in which the anchor portions 36 are configured with bottoms.

### Second Exemplary Embodiment

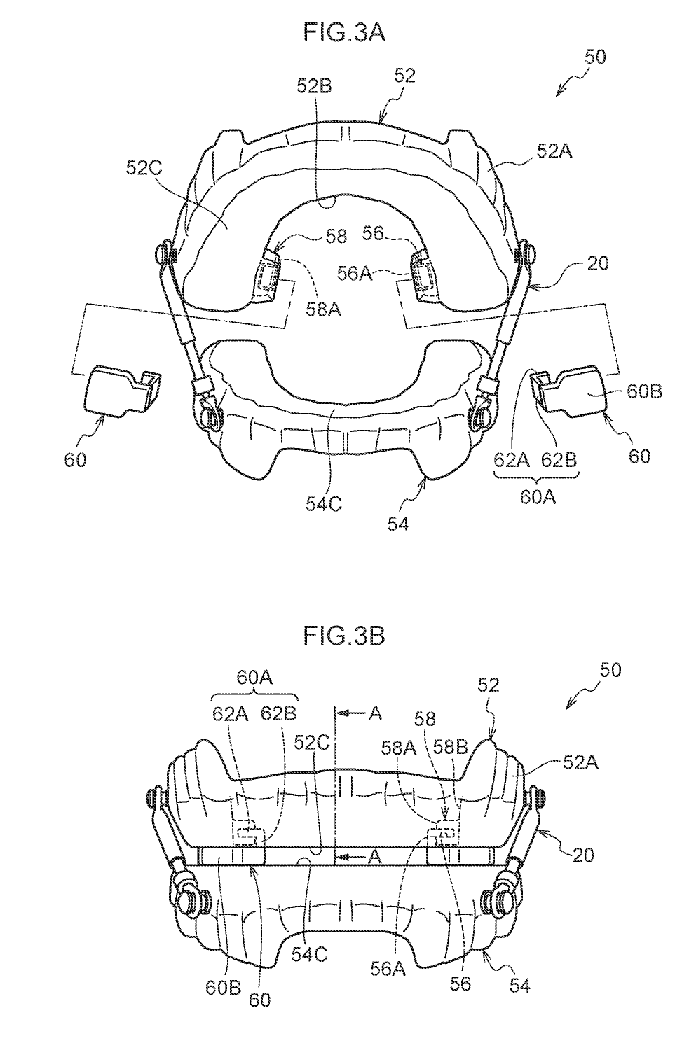

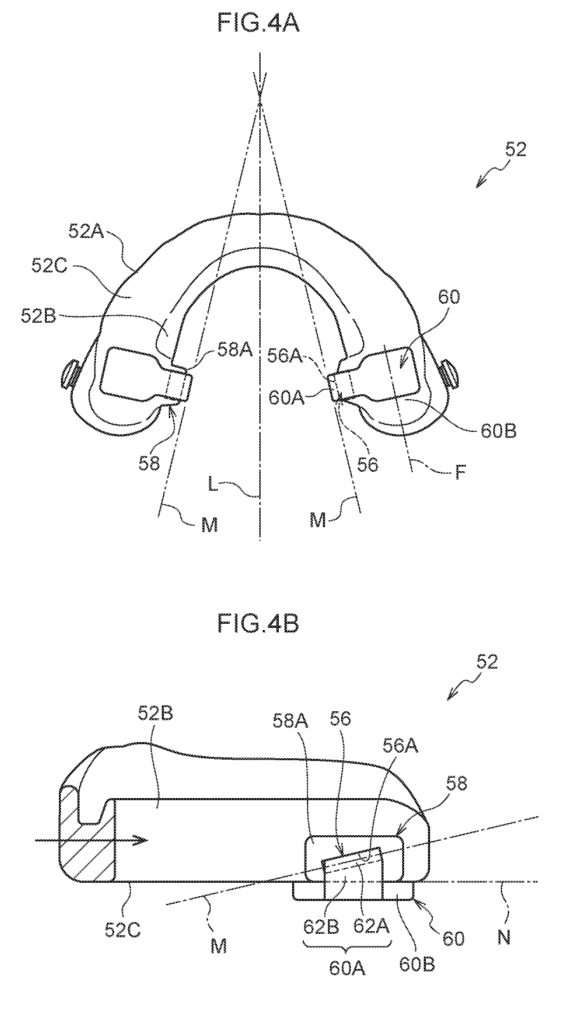

Next, explanation follows regarding a mouthpiece according to a second exemplary embodiment of the first aspect, with reference to FIG. 3A to FIG. 4B. Note that the same reference numerals are appended to configuration similar to that of the first exemplary embodiment, and explanation thereof will be omitted.

As illustrated in FIG. 3A and FIG. 3B, a mouthpiece 50 of the present exemplary embodiment, similarly to the mouthpiece 10 of the first exemplary embodiment, includes a maxillary piece 52 and a mandibular piece 54 coupled together by coupling members 20.

Respective anchor bases 58 including anchor portions 56 are formed so as to project from an inner wall face 52B at left and right sides (at tooth positions 6 to 7 in the present exemplary embodiment) of the maxillary piece 52 as viewed from a center of dentition. The anchor portions 56 are configured by substantially rectangular shaped indentations, and include elongated opening portions 56A at inner wall faces 58A of the anchor bases 58.

As illustrated in FIG. 4A, when the maxillary piece 52 is viewed from an opposing face 52C side, imaginary lines M along length directions of the opening portions 56A (namely, along the inner wall face 58A of the anchor bases 58) extend in directions approaching a center line L of the dentition of the maxillary piece 52 on progression from a posterior side toward an anterior side, and intersect with the center line L. Namely, the length directions of the opening portions 56A of the anchor portions 56 (the imaginary lines M) are not parallel to the center line L.

Furthermore, as illustrated in FIG. 4B, the imaginary lines M along the length direction of the opening portions 56A extend in directions approaching an imaginary plane N, that is parallel to an opposing face 52C of the maxillary piece 52, on progression from the anterior side toward the posterior side, and intersect with the imaginary plane N. Namely, the length directions of the opening portions 56A of the anchor portions 56 (the imaginary lines M) are not parallel to the imaginary plane N.

Note that the angles of the imaginary lines M illustrated in FIG. 4A and FIG. 4B are examples thereof. The imaginary lines M may each has any angle to the center line L and to the imaginary plane N as long as they are not parallel thereto. However, angles formed between each of the imaginary lines M and the center line L or the imaginary plane N are preferably from 5 degrees to 45 degrees, are more preferably from 5 degrees to 30 degrees, and are still more preferably from 5 degrees to 20 degrees.

As illustrated in FIG. 3A, vertical adjustment members 60 are mounted to the anchor portions 56. The vertical adjustment members 60 include L-shaped protrusions 60A, each serving as a mounting portion and including a horizontal portion 62A and a vertical portion 62B, and a clamped portion 60B that extends from one end of the vertical portion 62B of the protrusion 60A in a direction parallel to the extension direction of the horizontal portion 62A. A retention means such as a hooked portion may be provided at the horizontal portion 62A of the protrusion 60A at each of the vertical adjustment members 60 that are fitted into the opening portions 56A of the anchor portions 56 to maintain the state fitted into the anchor portions 56.

Similarly to the vertical adjustment members 40 of the first exemplary embodiment, as illustrated in FIG. 4A, in a state in which the vertical adjustment members 60 are mounted to the anchor portions 56, the protrusions 60A of the vertical adjustment members 60 are provided at an inner wall face side of a plane F bisecting the vertical adjustment member 60 between the inner wall face side and the outer wall face side. Namely, the vertical adjustment members 60 have an asymmetrical shape with respect to the plane F.

When the horizontal portions 62A of the protrusions 60A at the vertical adjustment members 60 are fitted into the opening portions 56A of the anchor portions 56, the clamped portions 60B of the vertical adjustment members 60 are mounted to the opposing face 52C of the maxillary piece 52. Thus in a closed mouth state, as illustrated in FIG. 3B, the clamped portions 60B of the vertical adjustment members 60 are clamped between the opposing face 52C of the maxillary piece 52 and an opposing face 54C of the mandibular piece 54. A gap therefore arises between the maxillary piece 52 and the mandibular piece 54.

According to the present exemplary embodiment, by fitting the horizontal portions 62A of the protrusions 60A of the vertical adjustment members 60 into the anchor portions 56 of the anchor bases 58 formed at the maxillary piece 52, the vertical adjustment members 60 can be easily anchored to the maxillary piece 52, while also suppressing dropout of the vertical adjustment members 60.

Moreover, when the mouthpiece 50 is being worn, air (namely, inhaled air and exhaled air) flows along the center line L and along the imaginary plane N between the maxillary piece 52 and the mandibular piece 54 each time the patient breaths. Thus in the present exemplary embodiment, the length directions of the opening portions 56A of the anchor portions 56 formed at the inner wall faces 58A of the anchor bases 58 are not parallel to the center line L and the imaginary plane N, namely are not parallel to the airflow direction.

This enables the protrusions 60A of the vertical adjustment members 60 fitted to the anchor portions 56 to be better suppressed from dropping out from the anchor portions 56 due to the airflow than in configurations in which the length directions of the opening portions 56A of the anchor portions 56 are parallel to the center line L and the imaginary plane N.

### Third Exemplary Embodiment

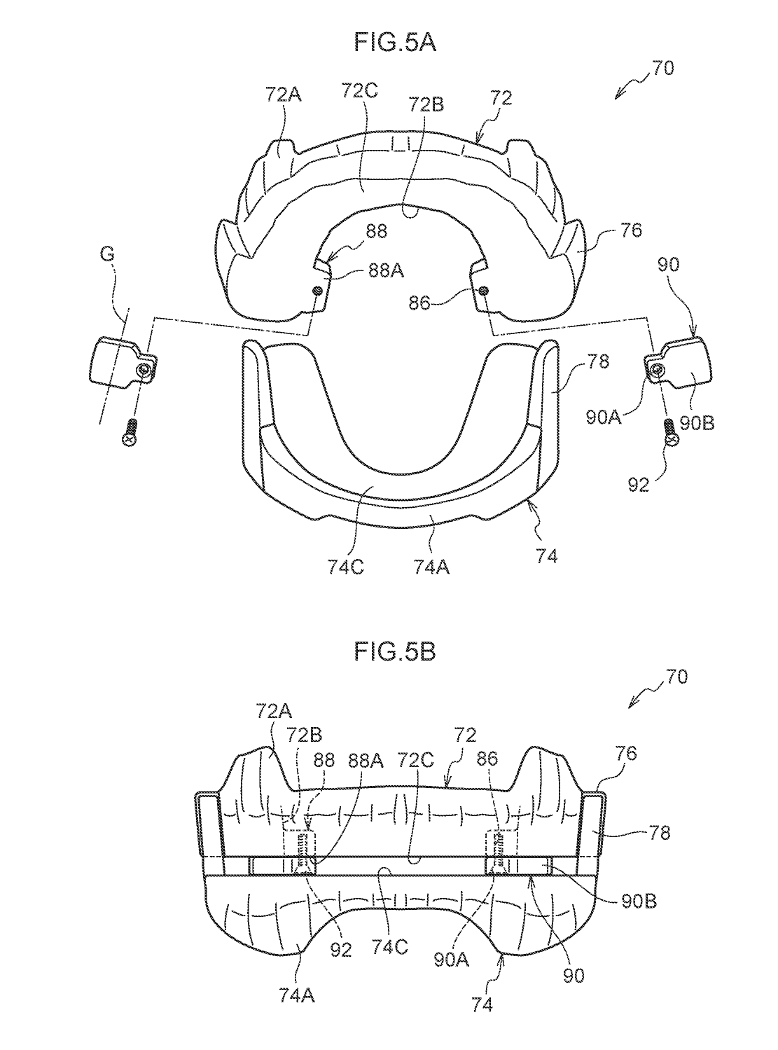

Next, explanation follows regarding a mouthpiece according to a third exemplary embodiment of the first aspect, with reference to FIG. 5A and FIG. 5B. Note that explanation of configuration similar to that of the first exemplary embodiment and the second exemplary embodiment will be omitted.

As illustrated in FIG. 5A, a mouthpiece 70 of the present exemplary embodiment includes a maxillary piece 72 and a mandibular piece 74. Stoppers 76 that each serves as a positioning member are formed projecting out from an outer wall face 72A of the maxillary piece 72. The stoppers 76 are formed integrally with the maxillary piece 72, and are provided at left and right posterior sides (at tooth positions 6 to 7 in the present exemplary embodiment) of the maxillary piece 72 as viewed from a center of dentition.

Wings 78 serving as positioning members are also formed projecting from an outer wall face 74A of the mandibular piece 74 and extending in a direction toward the maxillary piece 72. The wings 78 are configured from the same acrylic-based resin as the mandibular piece 74, and are adjustable in position between tooth positions 6 to 7 of the mandibular piece 74, that is disposed at an anterior side from the stopper 76, by use of non-illustrated screws.

When the mouthpiece 70 is being worn, as illustrated in FIG. 5B, movement of the wings 78 toward the posterior side is restricted by side faces of the wings 78 (namely, end faces on the posterior side of the dentition) abutting side faces of the stopper 76 (namely, end faces on the anterior side of the dentition). Namely, the mandibular piece 74 (and hence the mandible) is positioned by the wings 78 and the stoppers 76 so as not to move toward the posterior side with respect to the maxillary piece 72 (and hence the maxilla).

Moreover, respective anchor bases 88 are formed so as to project from an inner wall face 72B of the maxillary piece 72 at left and right sides (at tooth positions 6 to 7 in the present exemplary embodiment) as viewed from the center of dentition. Anchor portions 86 are respectively formed at the anchor bases 88. Note that the anchor portions 86 are configured by female threaded holes that open at lower faces of the anchor bases 88, namely opposing faces 88A opposing the mandibular piece 74. The opposing faces 88A of the anchor bases 88 are configured so as to lie in substantially the same plane as an opposing face 72C (namely the occlusal surface) of the maxillary piece 72.

As illustrated in FIG. 5A, vertical adjustment members 90 are mounted to the anchor bases 88. The vertical adjustment members 90 each includes a substantially rectangular shaped clamped portion 90B and a through hole 90A serving as a mounting portion and formed so as to penetrate through the clamped portion 90B. The vertical adjustment members 90 are fixed to the anchor bases 88 by screwing screws 92 that have been inserted through the through holes 90A into the anchor portions 86 of the anchor bases 88.

In a mounted state of the vertical adjustment members 90 to the anchor portions 86, the through holes 90A in the vertical adjustment members 90 are provided at an inner wall face side of a plane G bisecting the vertical adjustment member 90 between the inner wall face side and an outer wall face side. Namely, the vertical adjustment members 90 have asymmetrical shapes with respect to the plane G. Note that the vertical adjustment members 90 each preferably has left-right symmetry with respect to a plane bisecting the through hole 90A and the clamped portion 90B along an axial line of the through hole 90A.

The clamped portions 90B of the vertical adjustment members 90 are mounted to the opposing face 72C of the maxillary piece 72 when the vertical adjustment members 90 are fixed to the anchor bases 88. Thus in a closed mouth state, as illustrated in FIG. 5B, the clamped portions 90B of the vertical adjustment members 90 are clamped between the opposing face 72C of the maxillary piece 72 and an opposing face 74C of the mandibular piece 74. A gap therefore arises between the maxillary piece 72 and the mandibular piece 74.

In the present exemplary embodiment, the screws 92, that have been inserted through the through holes 90A of the vertical adjustment members 90, are screwed into the anchor portions 86 of the anchor bases 88 formed at the maxillary piece 72, whereby the vertical adjustment members 90 can be easily anchored to the maxillary piece 72, while also suppressing the vertical adjustment members 90 from dropping out.

### Other Exemplary Embodiments

The first aspect is not limited to the exemplary embodiments described above, and various other exemplary embodiments are possible within the scope of the present disclosure. Moreover, appropriate combinations may be made from the above first to third exemplary embodiments.

For example, whereas in the first to third exemplary embodiments, the anchor bases 38, 58, 88 were formed projecting out from the inner wall face 12B, 52B, 72B of the maxillary piece 12, 52, 72, the anchor bases 38, 58, 88 do not necessarily project out from the inner wall face 12B, 52B, 72B of the maxillary piece 12, 52, 72.

Moreover, the anchor bases 38, 58, 88 preferably do not project below the opposing face 12C, 52C, 72C of the maxillary piece 12, 52, 72. This enables obstruction of tongue movement by the anchor bases 38, 58, 88 to be suppressed. Furthermore, the anchor bases 38, 58, 88 may be formed at the inner wall face 14B, 54B, 74B of the mandibular piece 14, 54, 74, and may be formed at both the maxillary piece 12, 52, 72 and the mandibular piece 14, 54, 74.

Furthermore, the mounting portion configurations of the anchor portion 36, 56, 86 at the anchor bases 38, 58, 88 and the vertical adjustment members 40, 60, 90 are not limited to that of the above exemplary embodiments. For example, the anchor portions 56, 86 may be configured as through holes in the second and third exemplary embodiment, similarly to as in the first exemplary embodiment, and the anchor portions 36, 56 may be configured by female threaded holes in the first and second exemplary embodiments, similarly to as in the third exemplary embodiment.

Moreover, a rail groove serving as an anchor portion may be formed at the anchor bases 38, 58, 88, and a rail serving as a mounting portion to fit into the rail groove may be formed at the vertical adjustment members 40, 60, 90. Furthermore, a protrusion serving as an anchor portion may be formed at the anchor bases 38, 58, 88, and an indentation serving as a mounting portion into which the protrusion fits may be formed at the vertical adjustment members 40, 60, 90.

In addition, the vertical adjustment members 40, 60, 90 may be mounted to the anchor bases 38, 58, 88 by various methods, such as methods for anchoring using fastenings configured by a male member provided to one thereof and a female member configured to the other thereof.

Moreover, although in the first to third exemplary embodiments the gap between the maxillary piece 12, 52, 72 and the mandibular piece 14, 54, 74 is adjusted by the pair of vertical adjustment members 40, 60, 90, the number and position of the vertical adjustment members 40, 60, 90 (and the anchor bases 38, 58, 88) are not limited to those of the above exemplary embodiments.

Furthermore, the shape of the vertical adjustment members 40, 60, 90 may also be any shape. For example, asymmetries in jaw shape of the patient are correctable using the vertical adjustment members 40, 60, 90 by setting non-uniform thicknesses for the clamped portions 40B, 60B, 90B, or by using clamped portions 40B, 60B, 90B of different thicknesses in the pair of vertical adjustment members 40, 60, 90.

Moreover, in the second exemplary embodiment, the imaginary lines M along the length directions of the opening portions 56A of the anchor portions 56 are each not parallel to both the center line L and the imaginary plane N. However, the protrusions 60A of the vertical adjustment members 60 fitted into the anchor portions 56 can be suppressed from dropping out of the anchor portions 56 due to the airflow as long as the imaginary lines M are each set so as not to be parallel to at least one of the center line L or the imaginary plane N.

Moreover, although the mouthpiece 10, 50 is a push-type mouthpiece in the first and second exemplary embodiments, a pull-type configuration may be adopted in which the mandibular piece 14, 54 (mandible) is pulled forward by the coupling members 20, by setting the upper attachment portions 16 at the anterior side of the dentition from (at the front teeth side of) the lower attachment portions 18. Furthermore, the configuration of the coupling members 20 is also not limited to those of the exemplary embodiments described above, and any configuration may be adopted that is a configuration coupling the maxillary piece 12 and the mandibular piece 14 together so as to be capable of adjusting the length of the coupling portion.

Moreover, although in the third exemplary embodiment the stoppers 76 are formed at the maxillary piece 72 and the wings 78 are formed at the mandibular piece 74, the wings 78 may be formed at the maxillary piece 72 and the stoppers 76 formed at the mandibular piece 74.

Furthermore, the materials to configure the maxillary piece 12 and the mandibular piece 14, the upper attachment portions 16, the lower attachment portions 18, and the coupling members 20 are not limited to the examples of materials given in the first exemplary embodiment. For example, although the upper attachment portion 16, the lower attachment portion 18, and the coupling members 20 are made of metal therein, they may be configured from a plastic from the perspectives of enabling application to patients with metal allergies, achieving a saving in weight, while also retaining the strength of members, or reducing any feeling of discomfort inside the oral cavity.

Second Aspect

A mouthpiece according to a second aspect of the present disclosure includes a maxillary piece that is mounted to maxillary dentition, a mandibular piece that is mounted to mandibular dentition, a positioning member that positions the mandibular piece with respect to the maxillary piece, an anchor portion that includes a mount formed at at least one location or more at an outer periphery of at least one of the maxillary piece or the mandibular piece, or at an opposing face of the maxillary piece or the mandibular piece where they oppose each other, and a vertical adjustment member that is mounted to the mount of the anchor portion and is clamped between the maxillary piece and the mandibular piece.

In the second aspect, the mount of the anchor portion is formed at at least one location or more at the outer periphery of the maxillary piece or the mandibular piece or at the opposing face. There is accordingly a lower risk of being swallowed by the patient even if the vertical adjustment member were to drop out than configurations in which the mount is formed at an inner wall face side of the maxillary piece or the mandibular piece. This enables the vertical adjustment member to be made more compact, as the vertical adjustment member does not need to be enlarged.

Note that in the following second aspect, configuration referred to as “a projection” in the disclosure of Japanese Patent Application No. 2016-127904 is referred to as “an anchor base” herein, and configuration referred to as “a thick portion” therein is referred to as “a clamped portion” herein.

### First Exemplary Embodiment

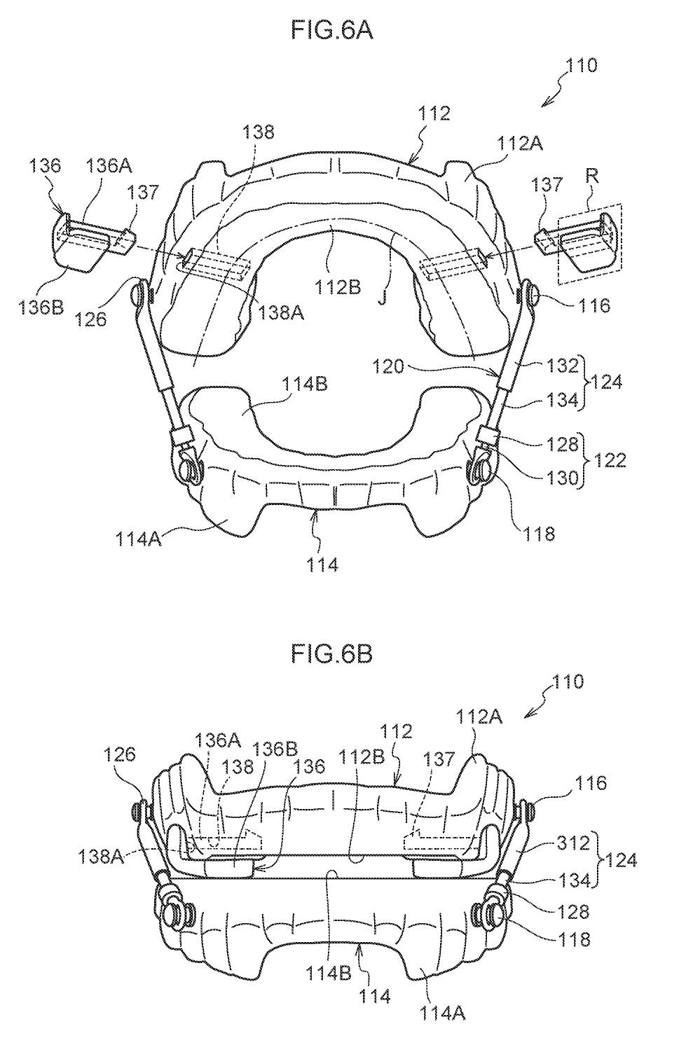

Explanation follows regarding a mouthpiece according to a first exemplary embodiment of the second aspect, with reference to FIG. 6A and FIG. 6B.

Similarly to the mouthpiece 10 of the first aspect, a mouthpiece 110 of the present exemplary embodiment is employed for sleep apnea syndrome. As illustrated in FIG. 6A and FIG. 6B, the mouthpiece 110 includes a maxillary piece 112 worn at maxillary dentition, and a mandibular piece 114 worn at mandibular dentition.

The maxillary piece 112 and the mandibular piece 114 are, for example, configured from an acrylic-based resin having a modulus of elasticity of approximately 2150 MPa in bending tests according to JIS T6501. Examples of materials for the maxillary piece 112 and the mandibular piece 114 also include polyester-based resins, polycarbonate-based resins, urethane-based resins, vinyl acetate-based resins, polyamide-based resins, and the like.

Metal upper attachment portions 116 are provided projecting from an outer wall face 112A of the maxillary piece 112, so as to project out from the outer wall face 112A at left and right posterior sides of a dentition (at tooth positions 6 to 7 in the present exemplary embodiment) as viewed from a center of the dentition. Metal lower attachment portions 118 are provided at an outer wall face 114A of the mandibular piece 114, so as to project out from the outer wall face 114A at left and right posterior sides of a dentition (at tooth positions 3-4 in the present exemplary embodiment) as viewed from the center of the dentition.

Moreover, the right upper attachment portion 116 and the right lower attachment portion 118, and the left upper attachment portion 116 and the left lower attachment portion 118, are respectively coupled together by metal coupling members 120 capable of pivoting. The coupling members 120 are positioning members to position such that the mandibular piece 114 does not move toward a posterior side with respect to the maxillary piece 112. The maxillary piece 112 and the mandibular piece 114 are coupled together by the pair of coupling members 120 so as to be capable of opening and closing.

In the mouthpiece 110 of the present exemplary embodiment, the upper attachment portion 116 is positioned at a posterior side from (namely, at the back teeth side of) the lower attachment portion 118 as viewed from the center of the dentition. Namely, the mouthpiece 110 is configured as a push-type mouthpiece that, when being worn, pushes the mandibular piece 114 (and hence the mandible) forward using the coupling members 120.

The coupling members 120 each includes a length adjustment mechanism 122, a sliding mechanism 124, and eyelets 126 attached to the upper attachment portion 116 and the lower attachment portion 118. The length adjustment mechanisms 122 each includes a nut 128 and a male screw 130 screwed into the nut 128. A screw amount of the male screws 130 is adjusted by rotating the nuts 128 to adjust the length of the coupling members 120 in a continuous manner.

The sliding mechanisms 124 each include a sleeve 132 and a rod 134. The coupling members 120 are able to follow the maxillary piece 112 and the mandibular piece 114 by the rods 134 sliding inside the sleeves 132 to match the movement of the maxillary piece 112 and the mandibular piece 114. A distance between the upper attachment portion 116 and the lower attachment portion 118 (a distance between respective centers) is adjusted by the coupling members 120 so as to be between about 18 mm and about 50 mm.

Anchor portions 138 are formed at an outer wall face 112A of the maxillary piece 112 at left and right sides (at tooth positions 4-5 in the present exemplary embodiment) as viewed from the center of the dentition. The anchor portions 138 each is formed as a substantially rectangular shaped through hole penetrating through to an inner wall face of the maxillary piece 112 from the outer wall face 112A thereof. Open ends at the outer wall face 112A side of the anchor portions 138 are configured as mounts 138A to which vertical adjustment members 136 are mounted.

The vertical adjustment members 136 are configured, for example, from a resin material, and include protrusions 136A that fit into the mounts 138A of the anchor portions 138, and clamped portions 136B that extend in a horizontal direction along extension directions of the protrusions 136A. Hooked portions 137 are formed at leading ends of the protrusions 136A. Note that the vertical adjustment members 136 have left-right symmetry with respect to a plane R bisecting each of the protrusions 136A along the extension direction of the protrusions 136A.

The clamped portions 136B each has a substantially rectangular profile and a thickness from about 1 mm to about 10 mm. An extension length (namely a width) of the clamped portions 136B is either substantially the same as a width of an opposing face 112B of the maxillary piece 112 that opposes the mandibular piece 114 (namely, a length from the outer wall face 112A to the inner wall face), or smaller than the width of the opposing face 112B.

When the protrusions 136A of the vertical adjustment members 136 are fitted into the mounts 138A of the anchor portions 138, the hooked portions 137 at the leading ends of the protrusions 136A are anchored inside the anchor portions 138 or at the inner wall face of the maxillary piece 112, preventing removal of the vertical adjustment members 136 from the anchor portions 138. Moreover, as illustrated in FIG. 6B, the clamped portions 136B of the vertical adjustment members 136 are mounted to the opposing face 112B of the maxillary piece 112.

Thus in a closed mouth state, the clamped portions 136B of the vertical adjustment members 136 are clamped between the opposing face 112B of the maxillary piece 112 and an opposing face 114B of the mandibular piece 114 opposing the maxillary piece 112. A gap therefore arises between the maxillary piece 112 and the mandibular piece 114.

According to the present exemplary embodiment, the protrusions 136A at the vertical adjustment members 136 fit into the anchor portions 138 of the maxillary piece 112, enabling the vertical adjustment members 136 to be easily anchored to the maxillary piece 112. Moreover, due to the vertical adjustment members 136 being detachably attached to the anchor portions 138 of the maxillary piece 112, the gap between the maxillary piece 112 and the mandibular piece 114 can be appropriately adjusted by replacing the vertical adjustment members 136 with others equipped with clamped portions 136B of a different thickness.

Specifically, by preparing plural vertical adjustment member 136 with different thicknesses of clamped portion 136B by 1 mm, the gap between the maxillary piece 112 and the mandibular piece 114 can be adjusted by 1 mm by selecting the vertical adjustment member 136 that matches a gap between the upper and lower teeth of a patient. Note that a configuration may be adopted in which a gap is not formed (namely, the thickness is 0 mm) by anchoring vertical adjustment members 136 that only include the protrusions 136A into the anchor portions 138.

Moreover, in the present exemplary embodiment there are two anchor portions 138 formed at the outer wall face 112A of the maxillary piece 112, and the gap between the maxillary piece 112 and the mandibular piece 114 is adjusted by the pair of vertical adjustment members 136. This enables play between the maxillary piece 112 and the mandibular piece 114 to be suppressed better than configurations in which the gap is adjusted using a single vertical adjustment member 136.

Moreover, in the present exemplary embodiment, the mounts 138A of the anchor portions 138 are each formed at the outer wall face 112A of the maxillary piece 112, namely, at outer sides of a line J passing through a width direction center of the opposing face 112B of the maxillary piece 112.

There is accordingly a lower risk of being swallowed by the patient even if the vertical adjustment members 136 were to drop out than configurations in which the mounts 138A are formed at the inner wall face of the maxillary piece 112. Furthermore, when the mouthpiece 110 is mounted, the vertical adjustment members 136 are sandwiched between the maxillary piece 112 and a cheek of the patient, enabling dropout of the vertical adjustment members 136 to be suppressed.

Moreover, in the present exemplary embodiment the anchor portions 138 are configured by through holes that pierce through from the outer wall face 112A of the maxillary piece 112 to the inner wall face thereof. Cleaning the anchor portions 138 is accordingly easier than configurations in which the anchor portions 138 are bottomed. Furthermore, hooked portions 137 are formed at the leading ends of the protrusions 136A at the vertical adjustment members 136, enabling the vertical adjustment members 136 to be suppressed from dropping out from the anchor portions 138.

Moreover, in the present exemplary embodiments, the vertical adjustment members 136 have left-right symmetry, enabling common vertical adjustment members 136 to be employed for anchoring to the anchor portions 138 at both the left and right sides of the maxillary piece 112. Note that, when necessary, fine adjustments to the shape of the vertical adjustment members 136 are possible by separate machining of the vertical adjustment members 136.

### Second Exemplary Embodiment

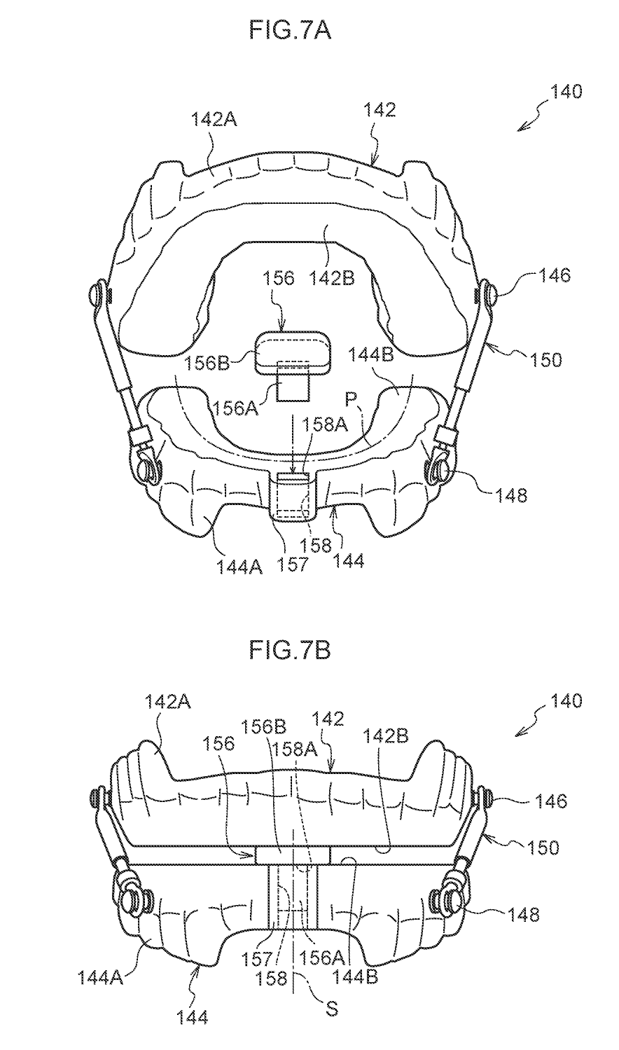

Explanation follows regarding a mouthpiece according to a second exemplary embodiment of the second aspect, with reference to FIG. 7A and FIG. 7B. Note that explanation will be omitted of configuration similar to that of the first exemplary embodiment.

As illustrated in FIG. 7A and FIG. 7B, in a mouthpiece 140 of the present exemplary embodiment, an anchor base 157 is formed at an outer wall face 144A at a center of a dentition (namely tooth position 1) of a mandibular piece 144 such that the anchor base 157 is projected further outer side than other portions of the outer wall face 144A. Note that the upper face of the anchor base 157 preferably does not project above an opposing face 144B of the mandibular piece 144, and more preferably lies in substantially the same plane as the opposing face 144B of the mandibular piece 144.

Moreover, an anchor portion 158 is formed at an upper face of the anchor base 157. The anchor portion 158 is a substantially rectangular shaped through hole piercing through from the upper face to a lower face of the anchor base 157. An open end of the anchor portion 158 at an upper face side of the anchor base 157 configures a mount 158A into which a vertical adjustment member 156 is mounted.

The vertical adjustment member 156 includes a protrusion 156A that fits into the mount 158A of the anchor portion 158, and a clamped portion 156B that extends in a direction orthogonal to an extension direction of the protrusion 156A. An extension direction length of the protrusion 156A of the vertical adjustment member 156 is shorter than a depth of the anchor portion 158 (namely, than a length from the upper face to the lower face of the anchor base 157). The vertical adjustment member 156 also has left-right symmetry with respect to a plane S bisecting the protrusion 156A along the extension direction of the protrusion 156A.

When the protrusion 156A of the vertical adjustment members 156 is fitted into the mount 158A of the anchor portion 158, as illustrated in FIG. 7B, the clamped portion 156B of the vertical adjustment member 156 is mounted to the opposing face 144B of the mandibular piece 144. Thus, in a closed mouth state, the clamped portion 156B of the vertical adjustment member 156 is clamped between an opposing face 142B of a maxillary piece 142 and the opposing face 144B of the mandibular piece 144. A gap therefore arises between the maxillary piece 142 and the mandibular piece 144.

In the present exemplary embodiment, similarly to in the first exemplary embodiment, the gap between the maxillary piece 142 and the mandibular piece 144 can be appropriately adjusted by replacing the vertical adjustment member 156 anchored at the anchor portion 158 of the mandibular piece 144 with another vertical adjustment member 156 equipped with a clamped portion 156B of a different thickness.

Moreover, according to the present exemplary embodiment, the anchor portion 158 is provided at the anchor base 157 formed at the outer wall face 144A of the mandibular piece 144. Moreover, the extension direction length of the protrusion 156A of the vertical adjustment member 156 is shorter than the depth of the anchor portion 158. This enables the protrusion 156A of the vertical adjustment member 156 to be prevented from interfering with the dentition when the vertical adjustment member 156 has been fitted into the anchor portion 158.

Note that the anchor portion 158 is formed at an outer side of a line P passing through a width direction center of the opposing face 144B of the mandibular piece 144. There is accordingly a lower risk of being swallowed by the patient even if the vertical adjustment member 156 were to dropout than configurations in which the anchor portion 158 was formed at an inner side of the line P passing through the center.

Moreover, in the present exemplary embodiment the anchor portion 158 is formed in a direction orthogonal to the opposing face 144B of the mandibular piece 144. The risk of the protrusion 156A of the vertical adjustment member 156 being removed from the anchor portion 158 of the mandibular piece 144 is accordingly lower than in configurations in which the anchor portion 158 is formed in a horizontal direction to the opposing face 144B of the mandibular piece 144, even in cases in which force is applied to the opposing face 144B in a horizontal direction (namely, a direction parallel to the opposing face 144B) by bruxism or the like.

Moreover, the gap between the maxillary piece 142 and the mandibular piece 144 is adjusted by the single vertical adjustment member 156. This enables the number of components to be reduced compared to configurations in which adjustment is made using plural vertical adjustment members 156.

### Third Exemplary Embodiment

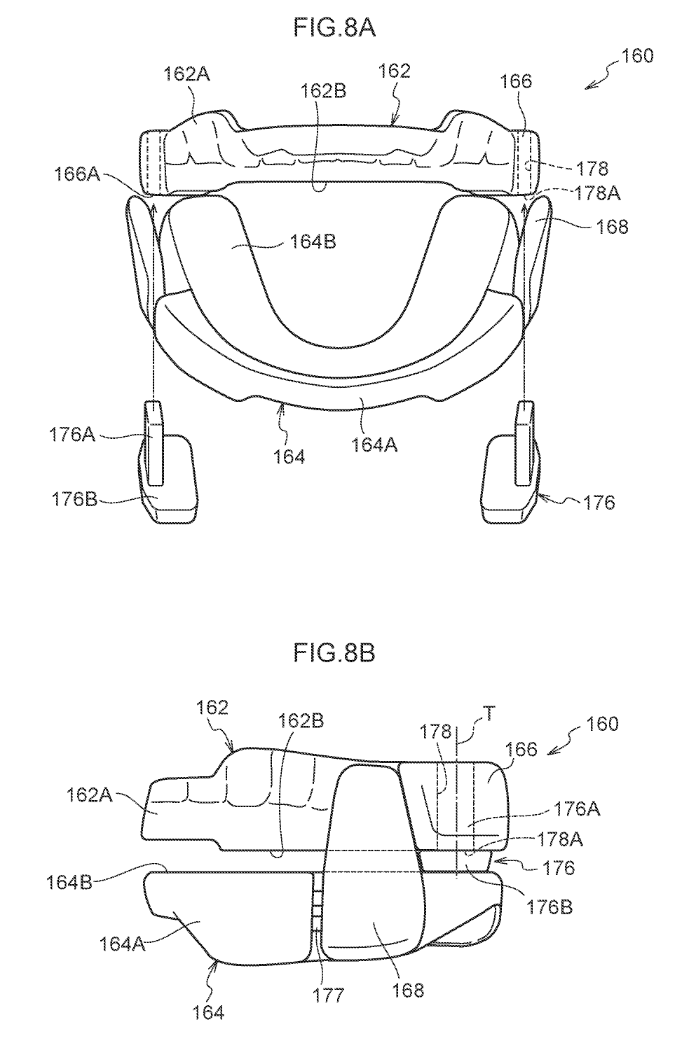

Next, explanation follows regarding a mouthpiece according to a third exemplary embodiment of the second aspect, with reference to FIG. 8A and FIG. 8B. Note that explanation will be omitted of configuration similar to that of the first exemplary embodiment and the second exemplary embodiment.

As illustrated in FIG. 8A and FIG. 8B, in a mouthpiece 160 of the present exemplary embodiment, stopper portions 166 serving as positioning members are formed projecting from an outer wall face 162A of a maxillary piece 162. The stopper portions 166 are formed integrally with the maxillary piece 162, and are provided at left and right posterior sides of a dentition (at tooth positions 6 to 7 in the present exemplary embodiment) of the maxillary piece 162 as viewed from a center of the dentition.

Wing portions 168 serving as positioning members are also formed projecting from an outer wall face 164A of a mandibular piece 164 and extending toward the maxillary piece 162. The wing portions 168 are configured from the same acrylic-based resin as the mandibular piece 164, and, by use of screws 177, are adjustable in position between tooth positions 6 to 7 of the mandibular piece 164, which is an anterior side from the stopper portions 166.

When the mouthpiece 160 is being worn, the wing portions 168 is restricted from moving toward a posterior side by side faces (namely, end faces at the posterior side of the dentition) of the wing portions 168 abutting side faces (namely, end faces at the anterior side of the dentition) of the stopper portions 166. Namely, the mandibular piece 164 (and hence the mandible) is positioned by the wing portions 168 and the stopper portions 166 so that the mandibular piece 164 does not move toward the posterior side with respect to the maxillary piece 162 (and hence the maxilla).

Moreover, anchor portions 178 are formed at lower faces 166A of the stopper portions 166 that oppose the mandibular piece 164. The anchor portions 178 are substantially rectangular shaped through holes penetrating through the stopper portions 166 from a lower face 166A to an upper face thereof. Open ends of the anchor portions 178 at a lower face 166A side of the stopper portions 166 configure mounts 178A into which vertical adjustment members 176 are mounted.

The vertical adjustment members 176 include protrusions 176A that fit into the mounts 178A of the anchor portions 178, and clamped portions 176B that extend in a direction orthogonal to an extension direction of the protrusions 176A. An extension direction length of the protrusions 176A of the vertical adjustment members 176 is shorter than a depth of the anchor portions 178 (namely, a length from the lower face 166A to the upper face of the stopper portions 166). The vertical adjustment members 176 each has left-right symmetry with respect to a plane T bisecting the protrusion 176A along the extension direction of the protrusion 176A.

When the protrusions 176A of the vertical adjustment members 176 are fitted into the mounts 178A of the anchor portions 178, as illustrated in FIG. 8B, the clamped portions 176B of the vertical adjustment members 176 are mounted to the lower face 166A of the stopper portions 166 and an opposing face 162B of the maxillary piece 162. Thus in a closed mouth state, the clamped portions 176B of the vertical adjustment members 176 are clamped between the opposing face 162B of the maxillary piece 162 and the opposing face 164B of the mandibular piece 164. A gap therefore arises between the maxillary piece 162 and the mandibular piece 164.

Note that in this situation, end faces at an anterior side of the dentition of the clamped portions 176B of the vertical adjustment members 176, together with the stopper portions 166, abut side faces (namely, end faces at a posterior side of the dentition) of the wing portions 168, restricting the wing portions 168 from moving toward the posterior side.

The present exemplary embodiment, similarly to the first exemplary embodiment and the second exemplary embodiment, enables the gap between the maxillary piece 162 and the mandibular piece 164 to be appropriately adjusted by replacing the vertical adjustment members 176 anchored to the anchor portions 178 of the stopper portions 166 with other vertical adjustment members 176 equipped with clamped portions 176B of a different thickness.

Moreover, in the present exemplary embodiment, due to forming the anchor portions 178 at the lower faces 166A of the stopper portions 166, the stopper portions 166 can be utilized as anchor portions for the vertical adjustment members 176. This enables the thickness and width of the maxillary piece 162 to be thinner than in configurations in which the anchor portions 178 are formed at the outer wall face 162A and the opposing face 162B of the maxillary piece 162, since additional space to form the anchor portions 178 is no longer required.

### Other Exemplary Embodiments

Note that the second aspect is not limited to the exemplary embodiments described above, and various other exemplary embodiments are possible within the scope of the present disclosure. Moreover, appropriate combinations may be made from the above first to third exemplary embodiments. For example, although the anchor portions 138 are configured by through holes in the first exemplary embodiment, the anchor portions 138 may be configured by bottomed indentations instead of through holes.

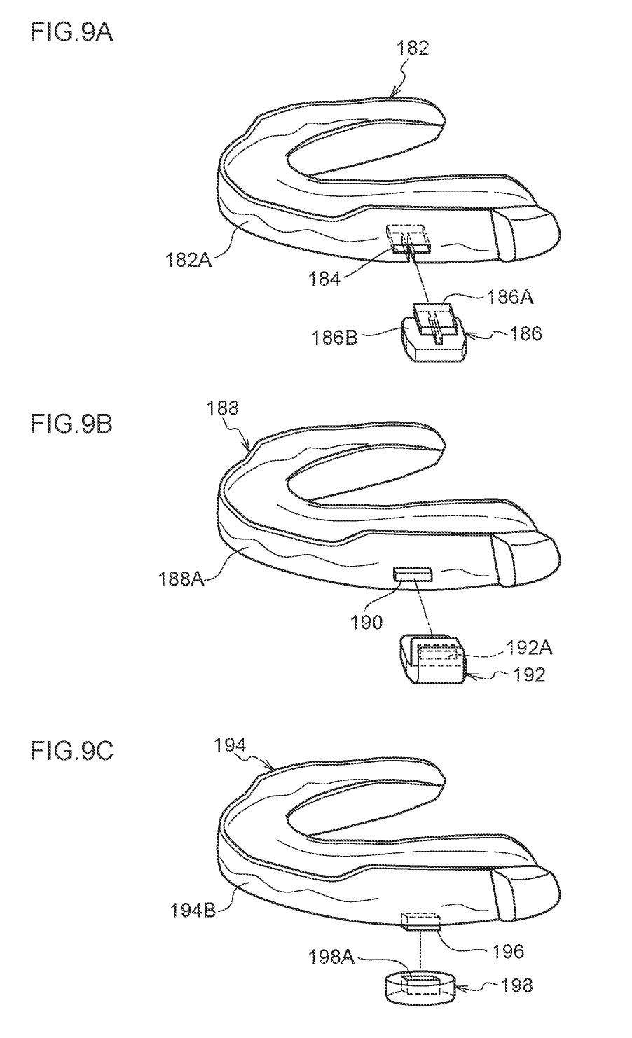

Moreover, as illustrated in FIG. 9A, rail grooves 184 serving as anchor portions may be formed at outer wall face 182A of a maxillary piece 182, and the vertical adjustment members 186 are anchored to the maxillary piece 182 by fitting rails 186A provided at upper faces of clamped portions 186B of vertical adjustment members 186 into the rail grooves 184.

In such cases, the vertical adjustment members 186 can be better suppressed from projecting out from the outer wall face 182A of the maxillary piece 182 than in the mouthpiece 110 of the first exemplary embodiment. Moreover, as illustrated in FIG. 9B, protrusions 190 serving as anchor portions may be formed at an outer wall face 188A of a maxillary piece 188, and the protrusions 190 of the maxillary piece 188 fitted into indentations 192A provided at vertical adjustment members 192.

Similarly, in the second exemplary embodiment, the anchor portion 158 is formed at the opposing face 144B of the mandibular piece 144 (namely, at the upper face of the anchor base 157), and the protrusion 156A provided at the vertical adjustment member 156 fitted therein. However, as illustrated in FIG. 9C, protrusions 196 serving as anchor portions may be formed at an opposing face 194B of a maxillary piece 194, and the protrusions 196 of the maxillary piece 194 fitted into indentations 198A provided at vertical adjustment members 198.

In addition, the vertical adjustment members 136 may be anchored to the maxillary piece 112 and the mandibular piece 114 by various methods, such as methods for using fastenings configured by a male member provided at one thereof and a female member configured at the other thereof, methods for using screw mechanisms configured by a male threaded portion provided at one thereof and a female threaded portion provided at the other thereof, or the like.

Moreover, although in the first exemplary embodiment the gap between the maxillary piece 112 and the mandibular piece 114 is adjusted using the pair of vertical adjustment members 136, a configuration may be adopted in which the vertical adjustment members 136 are coupled together as a single member, or are coupled together and integrated into a single unit.

Moreover, the vertical adjustment members 136 may have any shape as long as they have a configuration enabling the gap between the maxillary piece 112 and the mandibular piece 114 to be adjusted. Furthermore, the number and anchor positions of the vertical adjustment members 136 are not limited to those of the exemplary embodiment described above, and a configuration may be adopted in which three or more of the vertical adjustment members 136 are anchored to the maxillary piece 112 and the mandibular piece 114.

Moreover, in the third exemplary embodiment the anchor portions 178, to which the vertical adjustment members 176 are anchored, are formed at the stopper portions 166 serving as positioning members. However, the stopper portions 166 may themselves configure anchor portions, and a configuration may be adopted in which the vertical adjustment members 176 are directly anchored to the stopper portions 166.

Similarly, in the first and third exemplary embodiments, the coupling members 120 may themselves serve as positioning members, and the wing portions 168 may themselves serve as anchor portions. Specifically, for example, a configuration may be adopted in which the vertical adjustment members 136, 176 are directly anchored to the coupling members 120 and the wing portions 168 by providing through holes in the vertical adjustment members 136, 176, and inserting the coupling members 120 and the wing portions 168 through the through holes.

Moreover, in the first exemplary embodiment, the mounts 138A to which the vertical adjustment members 136 are mounted are configured only at open ends at the outer wall face 112A side of the maxillary piece 112. However, the mounts 138A to which the vertical adjustment members 136 are mounted may be formed at both the open ends on the outer wall face 112A side and open ends on an inner wall face side (not illustrated in the drawings).

Specifically, a configuration may be adopted in which a pair of protrusions 136A are formed at each of the vertical adjustment members 136, and each of the pairs of protrusions 136A are respectively fitted into the mounts 138A on the outer wall face 112A side and the inner wall face side of the maxillary piece 112.

Moreover, although the mouthpiece 110, 140 is a push-type mouthpiece in the first and second exemplary embodiments, a pull-type configuration may be adopted in which the mandibular piece 114, 144 (and hence the mandible) is pulled forward by the coupling members 120, 150 by setting the upper attachment portions 116, 146 at the anterior side (namely, at a front teeth side) from the lower attachment portions 118, 148.

Moreover, although in the third exemplary embodiment the stopper portions 166 are formed at the maxillary piece 162, and the wing portions 168 are formed at the mandibular piece 164, the wing portions 168 may be formed at the maxillary piece 162 and the stopper portions 166 may be formed at the mandibular piece 164.

Moreover, although the end faces on the anterior side of the clamped portions 176B of the vertical adjustment members 176 are positioned to abut the side faces (namely, the end faces at the posterior side) of the wing portions 168, they may be positioned so as not to abut the side faces (namely, end faces at the posterior side) of the wing portions 168. Furthermore, similarly to in the first aspect, the maxillary piece 112 and the mandibular piece 114, and the upper attachment portion 116, the lower attachment portion 118, and the coupling members 120 may be configured from a plastic.

Third Aspect

A mouthpiece according to a third aspect of the present disclosure includes a maxillary piece that is mounted to maxillary dentition, a mandibular piece that is mounted to mandibular dentition, a wing portion that is provided at an outer wall face of one of the maxillary piece or the mandibular piece and that extends in a direction toward the other of the maxillary piece or the mandibular piece, a stopper portion that is provided at an outer wall face of the other of the maxillary piece or the mandibular piece, and that restricts movement to a posterior side of dentition of the mandibular piece by abutting the wing portion, and a relative position adjustment member that is provided at the wing portion or the stopper portion to adjust a relative position of the maxillary piece and the mandibular piece.

In the third aspect, the wing portion or the stopper portion, that is provided with the relative position adjustment member, is strongly fixed to the maxillary piece or the mandibular piece. This enables the size of the relative position adjustment member itself to be made smaller, as well as enabling dropout of the relative position adjustment member to be suppressed.

“A relative position adjustment member” referred to herein is a member to adjust at least one of a relative position of the maxillary and mandibular pieces in an anterior-posterior direction or a relative position (i.e. gap) between the maxillary and mandibular pieces in a vertical direction, and encompasses “a vertical adjustment member” of the first aspect and the second aspect.

Note that in the following third aspect, configuration referred to as “a relative position adjustment portion” in the disclosure of Japanese Patent Application No. 2016-127905 is referred to as “a relative position adjustment member” herein, and configuration referred to as “a thick portion” therein is referred to as “a clamped portion” herein.

### First Exemplary Embodiment

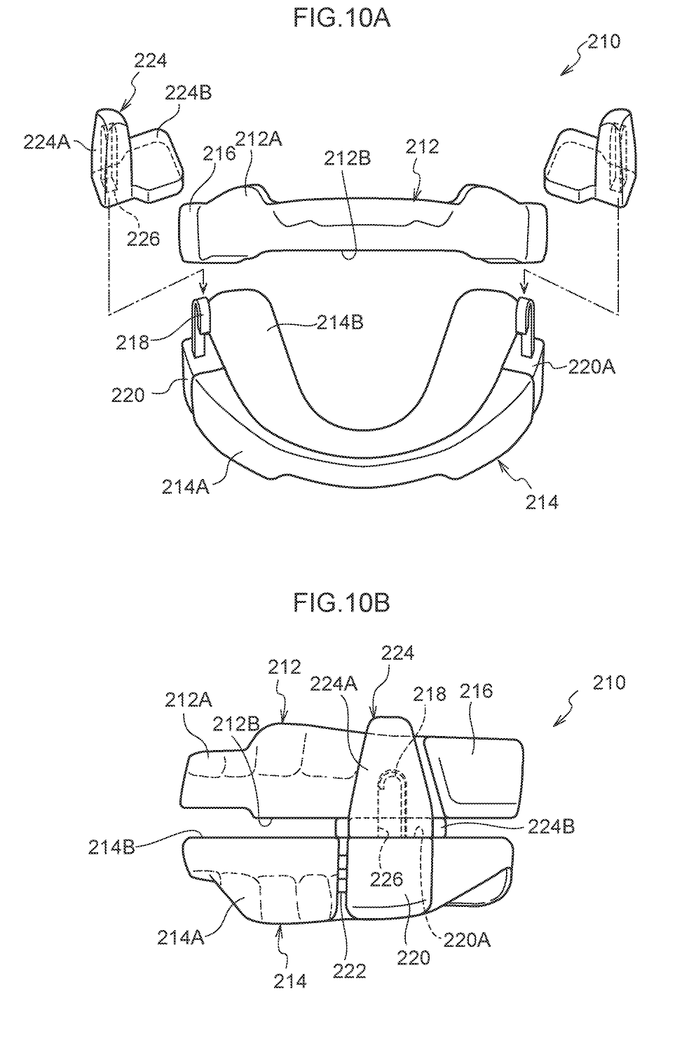

Explanation follows regarding a mouthpiece according to a first exemplary embodiment of the third aspect, with reference to FIG. 10A to FIG. 10B.

Similarly to in the first aspect and the second aspect, a mouthpiece 210 of the present exemplary embodiment is a mouthpiece employed for sleep apnea syndrome. As illustrated in FIG. 10A and FIG. 10B, the mouthpiece 210 includes a maxillary piece 212 worn at maxillary dentition, and a mandibular piece 214 worn at mandibular dentition. Note that the maxillary piece 212 and the mandibular piece 214 are, for example, configured from an acrylic-based resin having a modulus of elasticity of approximately 2150 MPa in bending tests according to JIS T6501.

Stopper portions 216 are formed projecting out from an outer wall face 212A of the maxillary piece 212. The stopper portions 216 are, for example, configured from the same acrylic-based resin as the maxillary piece 212, and are provided at left and right posterior sides (at tooth positions 6 to 7 in the present exemplary embodiment) of the maxillary piece 212 as viewed from a center of dentition.

Wing attachment portions 220 to which wing portions 224 are attached are formed at an outer wall face 214A of the mandibular piece 214. The wing attachment portions 220 are adjustable in position between tooth positions 6 to 7 of the mandibular piece 214, which is an anterior side from the stopper portions 216, by use of screws 222.

Moreover, upper faces of the wing attachment portions 220 (namely, faces opposing the maxillary piece 212) configure attachment faces 220A for wing portions 224, and projections 218 that project toward the maxillary piece 212 are provided at the attachment faces 220A. Note that the attachment faces 220A lie in substantially the same plane as an opposing face 214B of the mandibular piece 214.

The wing portions 224 are, for example, configured from the same acrylic-based resin as the mandibular piece 214. Moreover, the wing portions 224 each includes a wing 224A extending toward the maxillary piece 212, and a relative position adjustment member 224B that extends from a lower end of the wing 224A in a direction orthogonal to the extension direction of the wing 224A.

The relative position adjustment members 224B have substantially rectangular shapes and a thickness of from about 1 mm to about 10 mm. The extension length (namely, a width) of the relative position adjustment members 224B is substantially the same as a width (namely, a length from the outer wall face 214A to an inner wall face) of the opposing face 214B of the mandibular piece 214 opposing the maxillary piece 212, or smaller than the width of the opposing face 214B.

Moreover, attachment grooves 226 are formed at lower faces of the wing 224A (namely, faces opposing the mandibular piece 214). As illustrated in FIG. 10B, the wing portions 224 are detachably attached to the upper faces of the wing attachment portions 220 by the projections 218 with curved leading end portions fitting into the attachment grooves 226. Note that a configuration may be adopted in which a retention means such as hooked portions are provided at the projections 218 to maintain the fitted state thereof in the attachment grooves 226.

When the wing portions 224 are attached to the wing attachment portions 220, the relative position adjustment members 224B of the wing portions 224 are mounted to the opposing face 214B of the mandibular piece 214. Thus in a closed mouth state, the relative position adjustment members 224B are clamped between an opposing face 212B of the maxillary piece 212, opposing the mandibular piece 214, and the opposing face 214B of the mandibular piece 214. A gap therefore arises between the maxillary piece 212 and the mandibular piece 214.

Note that when the mouthpiece 210 is being worn, movement to a posterior side of the wing portions 224 is restricted by side faces (namely, end faces at a posterior side) of the wing portions 224 abutting side faces (namely, end faces at an anterior side) of the stopper portions 216. Namely, the mandibular piece 214 (and hence the mandible) is positioned by the wing portions 224 and the stopper portions 216 so that the mandibular piece 214 does not move to the posterior side with respect to the maxillary piece 212 (and hence the maxilla).

According to the present exemplary embodiment, the relative position adjustment members 224B are clamped between the maxillary piece 212 and the mandibular piece 214 thereby a gap being formed therebetween by the wing portions 224 that is formed integrally with the relative position adjustment members 224B and that is attached to the wing attachment portions 220 of the mandibular piece 214.

In such a configuration, the wing portions 224 are detachably attached to the wing attachment portions 220. This therefore enables the gap between the maxillary piece 212 and the mandibular piece 214 to be appropriately adjusted by replacing the wing portions 224 with others equipped with relative position adjustment members 224B of a different thickness.

Specifically, for example, the gap between the maxillary piece 212 and the mandibular piece 214 can be adjusted by 1 mm by preparing plural wing portions 224 equipped with relative position adjustment members 224B of different thicknesses by 1 mm, and then selecting a wing portions 224 that matches a gap between the upper and lower teeth of a patient.

Moreover, in the present exemplary embodiment, the wing portions 224 are respectively attached to the pair of wing attachment portions 220. Namely, the gap between the maxillary piece 212 and the mandibular piece 214 is adjusted by the pair of relative position adjustment members 224B. This means that play between the maxillary piece 212 and the mandibular piece 214 can be suppressed better than configurations in which the gap between the maxillary piece 212 and the mandibular piece 214 is adjusted using a single relative position adjustment member 224B.

Moreover, according to the present exemplary embodiment, the relative position adjustment members 224B are provided at the wing portions 224. This enables the thicknesses and widths of the maxillary piece 212 and the mandibular piece 214 to be thinner than in configurations in which the relative position adjustment members 224B are attached to an outer wall faces 212A, 214A and opposing faces 212B, 214B of the maxillary piece 212 and mandibular piece 214, since additional space to provide attachment portions for the relative position adjustment members 224B is no longer required.

Furthermore, due to the wing portions 224 being detachably attached to the wing attachment portions 220, vertical dimensions of the wing portions 224 can themselves be adjusted by attaching other wing portions 224 of a different vertical dimension to the attachment faces 220A of the wing attachment portions 220. Thus, for example as illustrated in FIG. 10B, the wing portions 224 are able to be abutted against the stopper portions 216 with certainty by setting the position of the upper end of the wing portions 224 higher than the position of the upper end of the stopper portions 216.

Note that in the present exemplary embodiment, the attachment faces 220A of the wing attachment portions 220 lie in substantially the same plane as the opposing face 214B of the mandibular piece 214. This makes for easier cleaning when maintenance is performed on the mouthpiece 210 by enabling cleaning of the attachment faces 220A of the wing attachment portions 220 to be performed at the same time as cleaning of the opposing face 214B of the mandibular piece 214.

### Second Exemplary Embodiment

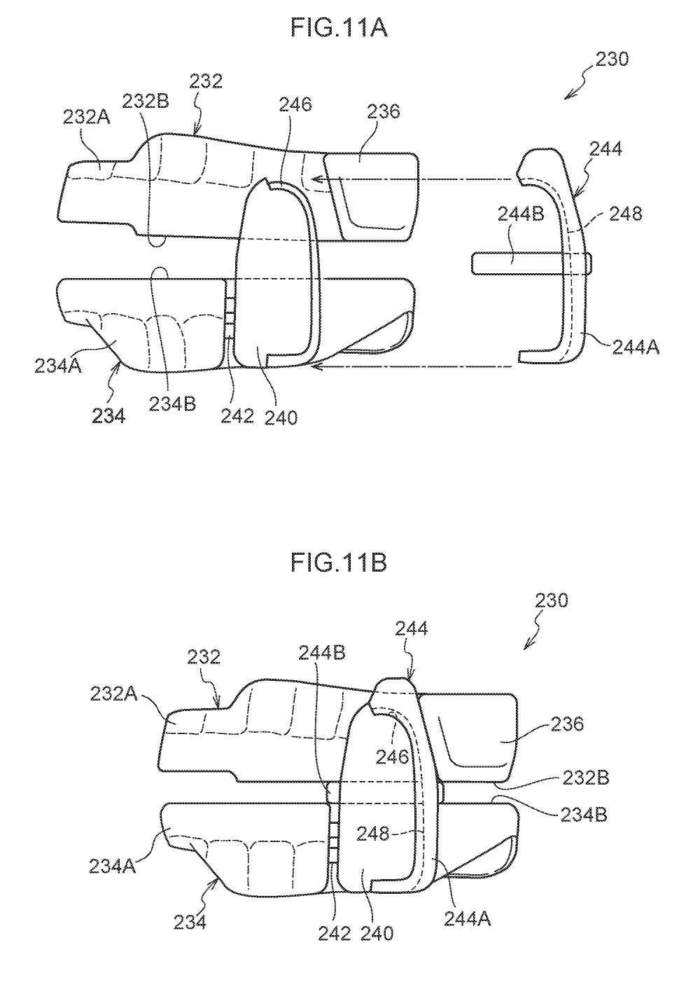

Next, explanation follows regarding a mouthpiece according to a second exemplary embodiment of the third aspect, with reference to FIG. 11A and FIG. 11B. Note that explanation will be omitted for configuration similar to that of the first exemplary embodiment.

In a mouthpiece 230 of the present exemplary embodiment, as illustrated in FIG. 11A and FIG. 11B, wing portions 240 are formed at an outer wall face 234A of a mandibular piece 234 so as to extend toward a maxillary piece 232. The wing portions 240 are adjustable in position between tooth positions 6 to 7 of the mandibular piece 234 that is at an anterior side from stopper portions 236 by use of screws 242.

Moreover, anchor portions 246, which are configured by fitting grooves are formed along an outer periphery of side faces (namely, end faces at a posterior side of dentition) of the wing portions 240 for attaching relative position adjustment members 244. The relative position adjustment members 244 each includes a cover member 244A that extends along the extension direction of the wing portions 240, and a clamped portion 244B that is provided at a substantially central portion in the extension direction of the cover member 244A and that extends in a direction substantially orthogonal to the cover member 244A.

Note that an extension length (namely, a vertical dimension) of the cover members 244A is substantially the same as an extension length (namely, a vertical dimension) of the wing portions 240, or longer (taller) than the extension length (namely, the vertical dimension) of the wing portions 240.

Fitting portions 248 are formed along an outer periphery of the side faces (namely, end faces at an anterior side of the dentition) of the cover members 244A. As illustrated in FIG. 11B, the cover members 244A of the relative position adjustment members 244 are detachably attached to the wing portions 240 by fitting portions 248 being fitted into anchor portions 246. Note that retention means such as hooked portions may be provided at the fitting portions 248 to maintain the fitted state thereof in the anchor portions 246.

When the cover members 244A of the relative position adjustment members 244 are attached to the wing portions 240, clamped portions 244B of the relative position adjustment members 244 are mounted to opposing faces 234B of the mandibular piece 234. Thus in a closed mouth state, the clamped portions 244B of the relative position adjustment members 244B are clamped between an opposing face 232B of the maxillary piece 232 and the opposing face 234B of the mandibular piece 234. A gap therefore arises between the maxillary piece 232 and the mandibular piece 234.

Note that when the mouthpiece 230 is being worn, movement to a posterior side of the wing portions 240 is restricted by side faces (namely, end faces at the posterior side of the dentition) of the cover members 244A of the relative position adjustment members 244 abutting side faces (namely, end faces at an anterior side of the dentition) of the stopper portions 236. In other words, the mandibular piece 234 (and hence the mandible) is positioned by the wing portions 240, the cover members 244A, and the stopper portions 236 so that the mandibular piece 234 does not move to the posterior side with respect to the maxillary piece 232 (and hence the maxilla).

According to the present exemplary embodiment, the relative position adjustment members 244 are detachably attached to the wing portions 240, thereby enabling the gap between the maxillary piece 232 and the mandibular piece 234 to be appropriately adjusted by replacing the relative position adjustment members 244 with others equipped with clamped portions 244B of a different thickness.

According to the present exemplary embodiment, movement of the wing portions 240 to the posterior side is restricted by the cover members 244A abutting the stopper portions 236. Thus the gap between the stopper portions 236 and the wing portions 240, namely a relative position of the maxillary piece 232 and the mandibular piece 234, can be adjusted by replacing the relative position adjustment members 244 with others that have different widths (namely, lengths from the anterior side end faces to the posterior side end faces) of the cover members 244A or different angles at the posterior side end faces abutting the stopper portions 236.

Furthermore, the vertical dimension of the wing portions 240 (and the cover members 244A) can be adjusted by replacing the relative position adjustment members 244 with others that have different extension lengths (namely, vertical dimensions) of the cover members 244A. This means that, for example as illustrated in FIG. 11B, the wing portions 240 (and the cover members 244A) can be made to abut the stopper portions 216 with certainty by setting the position of an upper end of the respective cover members 244A higher than the position of the upper end of the stopper portions 236.

Moreover, in the present exemplary embodiment, the relative position adjustment members 244 are clamped by the stopper portions 236 and the wing portions 240 by abutting the cover members 244A against the stopper portions 236 when the mouthpiece 230 is being worn. This enables the relative position adjustment members 244 to be suppressed from dropping out from the wing portions 240.

Furthermore, in the present exemplary embodiment, the wing portions 240 to which the relative position adjustment members 244 are attached are provided at the outer wall face 234A of the mandibular piece 234. There is accordingly a lower risk of being swallowed by the patient even if the relative position adjustment members 244 were to dropout when the mouthpiece 230 is being worn than in configurations in which the wing portions 240 are provided at the inner wall face.

### Third Exemplary Embodiment

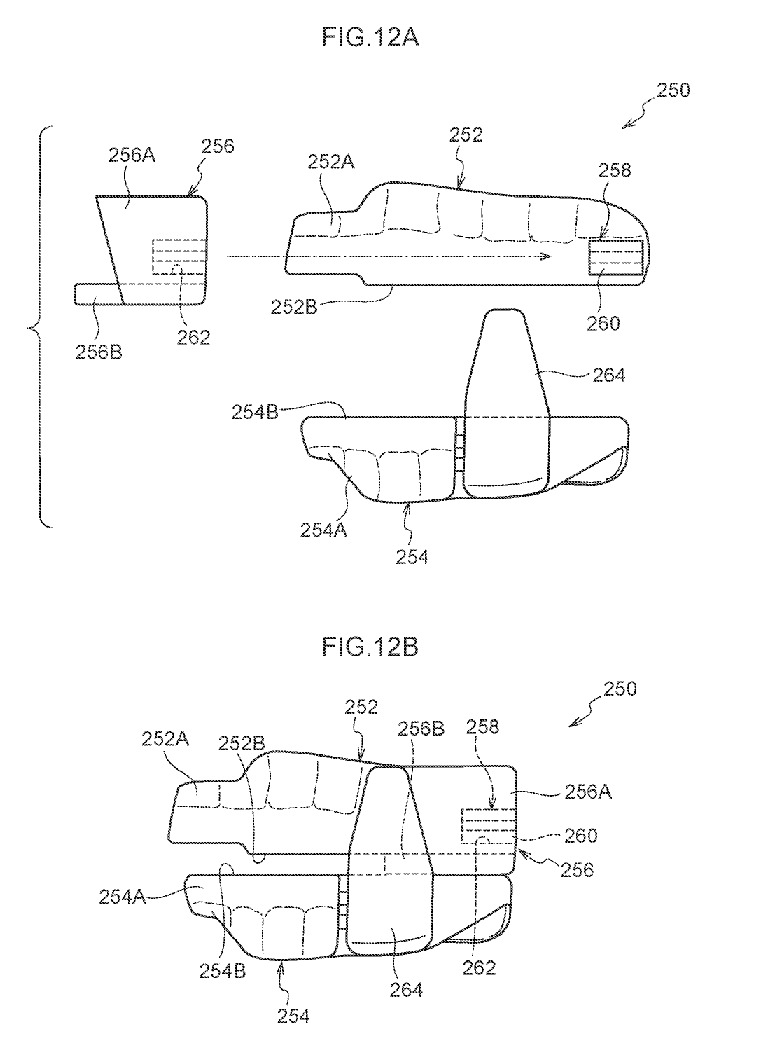

Explanation follows regarding a mouthpiece according to a third exemplary embodiment of the third aspect, with reference to FIG. 12A and FIG. 12B. Note that explanation of configuration similar to that of the first exemplary embodiment and the second exemplary embodiment will be omitted.

As illustrated in FIG. 12A and FIG. 12B, in a mouthpiece 250 of the present exemplary embodiment, stopper attachment portions 258 to which stopper portions 256 are attached are formed projecting from an outer wall face 252A of a maxillary piece 252 of the mouthpiece 250. The stopper attachment portions 258 are provided at the outer wall face 252A at the tooth positions 6 to 7 at the maxillary piece 252, and each includes a rail member 260 that has a T-shaped profile in cross-section and extends along the dentition.

The stopper portions 256 each includes a stopper 256A formed with a rail groove 262 at a posterior side of the dentition of the inner wall face, and a relative position adjustment member 256B that extends in a direction orthogonal to an inner wall face of the stopper 256A. Note that the rail groove 262 is provided along the inner wall face of each of the stoppers 256A, and one end of the rail groove 262 configures an opening at the posterior side of the stopper 256A.

As illustrated in FIG. 12B, the stopper portions 256 are detachably attached to the stopper attachment portions 258 by the rail members 260 being fitted into the rail grooves 262 of the stopper portions 256 from the posterior side. Note that a retention means may be provided at the rail members 260 to maintain the fitted state thereof in the rail grooves 262.

When the stopper portions 256 are attached to the stopper attachment portions 258, the relative position adjustment members 256B of the stopper portions 256 are mounted to an opposing face 252B of the maxillary piece 252. Thus in a closed mouth state, the relative position adjustment members 256B are clamped between the opposing face 252B of the maxillary piece 252 and an opposing face 254B of mandibular piece 254. A gap therefore arises between the maxillary piece 252 and the mandibular piece 254.

Note that when the mouthpiece 250 is being worn, movement to a posterior side of wing portions 264 is restricted by side faces (namely, end faces at a posterior side of the dentition) of wing portions 264 abutting side faces (namely, end faces at an anterior side of the dentition) of the stopper portions 256. In other words, the mandibular piece 254 (and hence the mandible) is positioned by the wing portions 264 and the stopper portions 256 so that the mandibular piece 254 does not move to the posterior side with respect to the maxillary piece 252 (and hence the maxilla).

In the present exemplary embodiment, the stopper portions 256, that are formed integrally with relative position adjustment members 256B, are detachably attached to the stopper attachment portions 258 of the maxillary piece 252. This enables the gap between the maxillary piece 252 and the mandibular piece 254 to be appropriately adjusted by replacing the stopper portions 256 with others equipped with relative position adjustment members 256B of a different thickness.

Moreover, in the present exemplary embodiment the relative position adjustment members 256B are provided at the stopper portions 256. This enables the thicknesses and widths of the maxillary piece 252 and the mandibular piece 254 to be thinner than in configurations in which the relative position adjustment members 256B are attached to the outer wall faces 252A, 254A and opposing faces 252B, 254B of the maxillary piece 252 and mandibular piece 254, since additional space to provide attachment portions for the relative position adjustment members 256B is not required.

Moreover, in the present exemplary embodiment, the stopper portions 256 are attached to the stopper attachment portions 258 by fitting the rail members 260, from the posterior side, into the rail grooves 262 formed at the posterior side of the stoppers 256A. This enables the stopper portions 256 to be easily attached to or detached from the stopper attachment portions 258 when the mouthpiece 250 is not being worn.

Furthermore, when the mouthpiece 250 is being worn, the stopper portions 256 can be suppressed from dropping out of the stopper attachment portions 258 due to the wing portions 264 abutting the end faces at the anterior side of the stopper portions 256 (namely, the stoppers 256A).

### Fourth Exemplary Embodiment

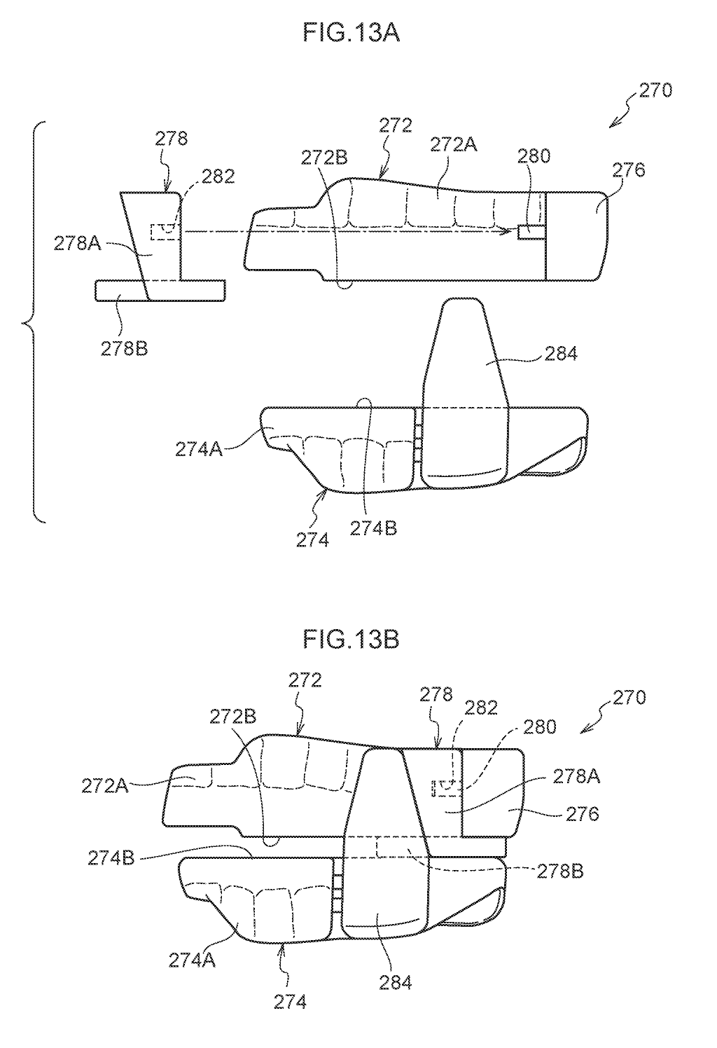

Next, explanation follows regarding a mouthpiece according to a fourth exemplary embodiment of the third aspect, with reference to FIG. 13A and FIG. 13B. Note that explanation will be omitted of configuration similar to that of the first to the third exemplary embodiments.

In a mouthpiece 270 of the present exemplary embodiment, as illustrated in FIG. 13A and FIG. 13B, stopper portions 276 are formed integrally to an outer wall face 272A of a maxillary piece 272. Moreover, anchor portions 280, configured by rectangular projections to which relative position adjustment members 278 are attached to, are formed projecting out to an anterior side from side faces (namely, end faces at an anterior side of the dentition) of the stopper portions 276.

The relative position adjustment members 278 include cover members 278A having substantially the same vertical dimension as a vertical dimension of the stopper portions 276 (namely, a width of an outer wall face 272A of the maxillary piece 272), and clamped portions 278B that extend from lower ends of the cover members 278A in directions orthogonal to the cover members 278A.

A rectangular shaped attachment groove 282 is formed at a side face at a posterior side of each of the cover members 278A. As illustrated in FIG. 13B, the cover members 278A of the relative position adjustment members 278 are detachably attached to the stopper portions 276 by the anchor portions 280 fitting into the attachment grooves 282. Note that a retention means such as a hooked portion may be provided at the anchor portions 280 to maintain the fitted state of the anchor portions 280 to the attachment grooves 282.

Moreover, through holes serving as anchor portions may be provided in the sides of the stopper portion 276, and projections may be provided at the sides of the cover member 278A to fit into the through holes. Furthermore, both through holes and projections serving as anchor portions may be provided at the stopper portions 276, and projections may be provided at the cover members 278A to fit into the through holes of the stopper portions 276 and through holes may be provided at the cover members 278A into which the projections of the stopper portions 276 fit.

The clamped portions 278B of the relative position adjustment members 278 are mounted to an opposing face 272B of the maxillary piece 272 by attaching the cover members 278A of the relative position adjustment members 278 to the stopper portions 276. Thus in a closed mouth state, the clamped portions 278B of the relative position adjustment members 278 are clamped between the opposing face 272B of the maxillary piece 272 and an opposing face 274B of a mandibular piece 274. A gap therefore arises between the maxillary piece 272 and the mandibular piece 274.

Note that when the mouthpiece 270 is being worn, movement to a posterior side of wing portions 284 is restricted by side faces at an anterior side of the cover members 278 abutting side faces at posterior side of the wing portions 284. In other words, the mandibular piece 274 (and hence the mandible) is positioned by the wing portions 284, the cover members 278A, and the stopper portion 276 so that the mandibular piece 274 does not move to the posterior side of the dentition relative to the maxillary piece 272 (and hence the maxilla).

In the present exemplary embodiment, the relative position adjustment members 278 are detachably attached to the stopper portions 276. This enables the gap between the maxillary piece 272 and the mandibular piece 274 to be appropriately adjusted by replacing the relative position adjustment members 278 with others equipped with clamped portions 278B of a different thickness.

Moreover, according to the present exemplary embodiment, movement to the posterior side of the wing portions 284 is restricted by the cover members 278A abutting the wing portions 284. This enables the gap between the stopper portions 276 and the wing portions 284, namely the relative position of the maxillary piece 272 and the mandibular piece 274, to be adjusted by replacing the relative position adjustment members 278 with others having a different width (namely, a length from end faces at the anterior side to end faces at the posterior side) of the cover members 278A or different angles at the end faces at the anterior side that abut the wing portions 284.

Moreover, in the present exemplary embodiment, the relative position adjustment members 278 are clamped by the stopper portions 276 and the wing portions 284 due to the cover members 278A abutting the wing portions 284 when the mouthpiece 270 is being worn. This enables the relative position adjustment members 278 to be suppressed from dropping out from the wing portions 284.

Other Exemplary Embodiments

Note that the third aspect is not limited to the exemplary embodiments described above, and various other exemplary embodiments are possible within the scope of the present disclosure. Moreover, appropriate combinations can be made from the first to the fourth exemplary embodiments.

For example, in the second exemplary embodiment, the relative position adjustment members 244 include the cover members 244A and the clamped portions 244B, the relative position of the maxillary piece 232 and the mandibular piece 234 is adjusted in the anterior-posterior dentition direction by the cover members 244A, and the gap between the maxillary piece 232 and the mandibular piece 234 is adjusted by the clamped portions 244B.

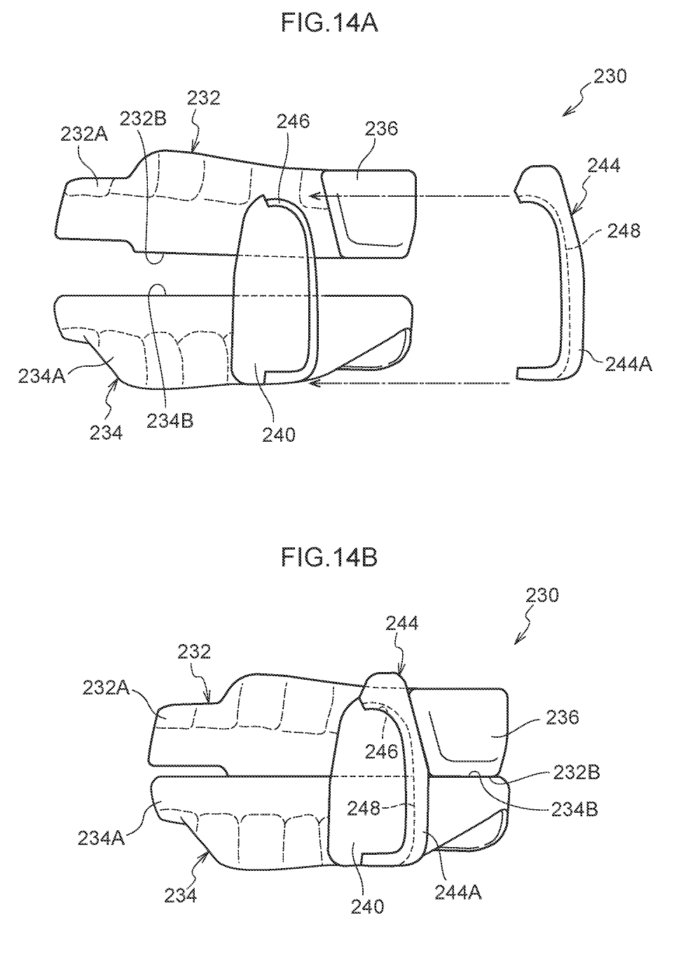

However, as illustrated in FIG. 14A and FIG. 14B, the relative position adjustment members 244 may include the cover members 244A alone. This enables the relative position of a maxillary piece 232 and a mandibular piece 234 to be adjusted only in the anterior-posterior direction of the dentition by replacing the relative position adjustment members 244 with others equipped with cover members 244A of a different width or a different angle at end faces at posterior sides that abut the stopper portions 236. Note that in such cases the screws 242 to adjust the position of the wing portions 240 do not need to be provided.

Similarly, in the fourth exemplary embodiment, the relative position adjustment members 278 include the cover members 278A and the clamped portions 278B, the relative position of the maxillary piece 272 and the mandibular piece 274 is adjusted in the anterior-posterior direction by the cover members 278A, and the gap between the maxillary piece 272 and the mandibular piece 274 is adjusted by the clamped portions 278B.

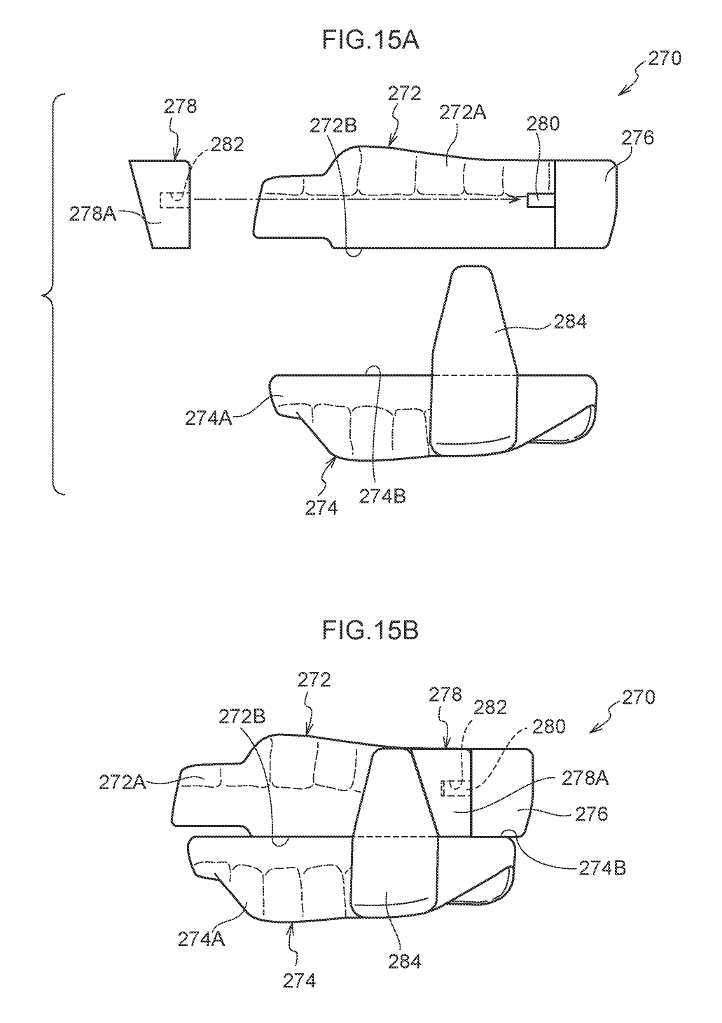

However, as illustrated in FIG. 15A and FIG. 15B, the relative position adjustment members 278 may include the cover members 278A alone. This enables the relative position of the maxillary piece 272 and a mandibular piece 274 to be adjusted only in the anterior-posterior direction by replacing the relative position adjustment members 278 with others equipped with cover members 278A of a different width or a different angle at the end faces at anterior sides that abut the wing portions 284.

Moreover, in the first exemplary embodiment, the wing portions 224 are attached to the wing attachment portions 220 by fitting the projections 218 of the wing attachment portions 220 into the attachment grooves 226 of the wings 224A. However, the wing portions 224 may be attached to the wing attachment portions 220 using any method as long as the wing portions 224 are detachably attached to the wing attachment portions 220. For example, projections may be provided at the wing 224A side and through holes or the like into which the projections fit provided at the wing attachment portions 220 side.

Similarly, the attachment method of the relative position adjustment members 244 to the wing portions 240 in the second exemplary embodiment, the attachment method of the stopper portion 256 to the stopper attachment portion 258 in the third exemplary embodiment, and the attachment method of the relative position adjustment members 278 to the cover members 278A in the fourth exemplary embodiment are not limited to those of the exemplary embodiments described above.

Specifically, for example in the second exemplary embodiment, a configuration may be adopted in which the relative position adjustment members 244 are directly anchored to the wing portions 240 by providing through holes that penetrate in the thickness direction through the clamped portions 244B of the relative position adjustment members 244, and inserting the wing portions 240 through the through holes.

Moreover, although in the first to fourth exemplary embodiments described above, the position of the wing portions 224, 240, 264, 284 are adjustable by use of the screws 242, the position of the stopper portions 216, 236, 256, 276 may be made adjustable instead of the wing portions 224, 240, 264, 284.

Furthermore, the positions (namely, angles) of the wing portions 224, 240, 264, 284 or the stopper portions 216, 236, 256, 276 may be made adjustable by rotating the wing portions 224, 240, 264, 284 or the stopper portions 216, 236, 256, 276 with respect to the maxillary piece 212, 232, 252, 272 or the mandibular piece 214, 234, 254, 274.

Moreover, in the first to the fourth exemplary embodiments, the stopper portions 216, 236, 256, 276 are provided at the maxillary piece 212, 232, 252, 272, and the wing portions 224, 240, 264, 284 are provided at the mandibular piece 214, 234, 254, 274. However, the wing portions 224, 240, 264, 284 may be provided at the maxillary piece 212, 232, 252, 272, and the stopper portions 216, 236, 256, 276 provided at the mandibular piece 214, 234, 254, 274.

The shape of the stopper portions 216, 236, 256, 276 may, as with the wing portions 224, 240, 264, 284, be shaped so as to extend in the direction of the other piece (the maxillary piece or the mandibular piece) that is not provided with the stopper portions 216, 236, 256, 276.

The entire disclosures of Japanese Patent Application Nos. 2016-127904 and 2016-127905 filed Jun. 28, 2016 are incorporated by reference in this specification, and entire disclosure of Japanese Patent Application No. 2016-224519 filed Nov. 17, 2016 is incorporated by reference in this specification.

All publications, patent applications and technical standards mentioned in the present specification are incorporated by reference in the present specification to the same extent as if each individual publication, patent application, or technical standard was specifically and individually indicated to be incorporated by reference.

1. A mouthpiece comprising:
a maxillary piece adapted to be mounted to maxillary dentition;
a mandibular piece adapted to be mounted to mandibular dentition;
a positioning member positioning the mandibular piece with respect to the maxillary piece;
an anchor base including an anchor portion, and formed at an inner wall face of at least one of the maxillary piece or the mandibular piece; and
a vertical adjustment member including a mounting portion mounted to the anchor portion of the anchor base, and a clamped portion clamped between the maxillary piece and the mandibular piece.

2. The mouthpiece of claim 1, wherein the anchor base is formed at a posterior side of dentition of at least one of the maxillary piece or the mandibular piece.

3. The mouthpiece of claim 1, wherein, in a mounted state of the mounting portion of the vertical adjustment member to the anchor portion of the anchor base, the vertical adjustment member has an asymmetrical shape with respect to a plane bisecting the vertical adjustment member between an inner wall face side and an outer wall face side of the maxillary piece or the mandibular piece.

4. The mouthpiece of claim 3, wherein, in a mounted state of the mounting portion of the vertical adjustment member to the anchor portion of the anchor base, the mounting portion is provided at the vertical adjustment member at the inner wall face side of the maxillary piece or the mandibular piece.

5.-9. (canceled)

10. A mouthpiece comprising:
a maxillary piece adapted to be mounted to maxillary dentition;
a mandibular piece adapted to be mounted to mandibular dentition;
a positioning member positioning the mandibular piece with respect to the maxillary piece;
an anchor portion including a mount and formed at at least one location or more at an outer periphery of at least one of the maxillary piece or the mandibular piece, or on an opposing face of the maxillary piece or the mandibular piece where they oppose each other; and
a vertical adjustment member mounted to the mount of the anchor portion and clamped between the maxillary piece and the mandibular piece.

11. The mouthpiece of claim 10, wherein the mount is formed at at least one location or more at an outer side of a line that extends along a width direction center of the opposing face.

12. The mouthpiece of claim 10, wherein an anchor base is formed at an outer wall face of the maxillary piece or the mandibular piece so as to project further to an outer side than other portions of the outer wall face, and the anchor portion is formed at the anchor base.

13. The mouthpiece of any one of claim 10, wherein:
the positioning member is attached at a posterior side of dentition of the maxillary piece and the mandibular piece; and
the anchor portion is formed at at least one location or more at an anterior side of the dentition from the positioning member at the maxillary piece or the mandibular piece.

14. The mouthpiece of claim 10, wherein:
the positioning member includes
a wing portion that is formed at an outer wall face of one of the maxillary piece or the mandibular piece and that extends in a direction toward another of the maxillary piece or the mandibular piece, and
a stopper portion that is formed at an outer wall face of the other of the maxillary piece or the mandibular piece at a position at a posterior side of the dentition Patent from the wing portion and that restricts movement of the wing portion to the posterior side; and
the mount of the anchor portion is formed at at least one location or more at a face of the stopper portion that opposes the maxillary piece or the mandibular piece.

15. The mouthpiece of any one of claim 10, wherein:
the anchor portion is configured as a through hole; and
the vertical adjustment member includes a protrusion, a length of the protrusion in an extension direction thereof being shorter than a depth of the through hole, and the protrusion being fit into the through hole.

16. The mouthpiece of claim 15, wherein the vertical adjustment member has left-right symmetry with respect to a plane bisecting the protrusion along the extension direction of the protrusion.

17. The mouthpiece of claim 15, wherein a hooked portion employed for retention is formed at a leading end of the protrusion.

18. A mouthpiece comprising:
a maxillary piece adapted to be mounted to maxillary dentition;
a mandibular piece adapted to be mounted to mandibular dentition;
a wing portion provided at an outer wall face of one of the maxillary piece or the mandibular piece and extending in a direction toward another of the maxillary piece or the mandibular piece;
a stopper portion provided at an outer wall face of the other of the maxillary piece or the mandibular piece, and restricting movement to a posterior side of the dentition of the mandibular piece by the stopper portion abutting the wing portion; and
a relative position adjustment member provided at the wing portion or the stopper portion so as to adjust a relative position of the maxillary piece and the mandibular piece.

19. The mouthpiece of claim 18, wherein:
the wing portion is provided at an outer wall face of the mandibular piece and extends toward the maxillary piece; and
the stopper portion is provided at an outer wall face of the maxillary piece at a posterior side of the dentition from the wing portion.

20. The mouthpiece of claim 18, wherein at least a portion of the relative position adjustment member is clamped between the maxillary piece and the mandibular piece.

21. The mouthpiece of claim 20, wherein:
the relative position adjustment member is formed integrally to the wing portion; and
the wing portion is detachably attached to a wing attachment portion provided at the maxillary piece or the mandibular piece.

22. The mouthpiece of claim 21, wherein the wing attachment portion is provided at an outer wall face of the maxillary piece or the mandibular piece.

23. The mouthpiece of claim 21, wherein an attachment face for the wing portion at the wing attachment portion lies in substantially a same plane as an opposing face of the maxillary piece that opposes the mandibular piece or an opposing face of the mandibular piece that opposes the maxillary piece.

24. The mouthpiece of claim 18, wherein:
the relative position adjustment member is detachably attached to an anchor portion of the wing portion; and
the wing portion is formed integrally to the maxillary piece or the mandibular piece.

25. The mouthpiece of claim 20, wherein:
the relative position adjustment member is formed integrally to the stopper portion; and
the stopper portion is detachably attached to a stopper attachment portion provided at the maxillary piece or the mandibular piece.

26. The mouthpiece of claim 25, wherein:
the stopper attachment portion includes a rail member provided at an outer wall face of the maxillary piece or the mandibular piece; and
a rail groove that the rail member fits into is formed at the stopper portion.

27. The mouthpiece of claim 18, wherein:
the relative position adjustment member is detachably attached to an anchor portion of the stopper portion; and
the stopper portion is formed integrally to the maxillary piece or the mandibular piece.

28. The mouthpiece of claim 24 wherein:
the anchor portion is provided at an abutting face of the wing portion abutting the stopper portion, or at an abutting face of the stopper portion abutting the wing portion; and
at least a portion of the relative position adjustment member is clamped by the wing portion and the stopper portion.
